(19) United States
(12) Patent Application Publication
(10) Pub. No.: US 2014/0358053 A1
(43) Pub. Date: Dec. 4, 2014

(54) POWER ASSISTED ORTHOSIS WITH HIP-KNEE SYNERGY

(71) Applicant: Case Western Reserve University, Cleveland, OH (US)

(72) Inventors: Ronald J. Triolo, Cleveland, OH (US); Rudolf Kobetic, Cleveland, OH (US); Curtis S. To, Cleveland, OH (US); Musa L. Audu, Cleveland, OH (US); Thomas C. Bulea, Cleveland, OH (US); Roger D. Quinn, Cleveland, OH (US); Mark Nandor, Cleveland Heights, OH (US)

(73) Assignee: Case Western Reserve University, Cleveland, OH (US)

(21) Appl. No.: 14/293,610

(22) Filed: Jun. 2, 2014

Related U.S. Application Data
(60) Provisional application No. 61/829,383, filed on May 31, 2013.

(51) Int. Cl. A61H 3/00; A61F 5/01

(52) U.S. Cl. CPC A61H 3/00; A61F 5/0102; A61F 5/0125; A61F 2005/0134; A61F 2005/0169; A61H 2003/001
USPC 602/16; 602/26; 602/19

(57) ABSTRACT

A power-assisted orthosis is shown and described. The power assisted orthosis may include a knee orthosis adapted to be secured to at least one of a user's knee joint, a hip orthosis adapted to be secured to a user's hip joints, and a first actuator attached with the knee orthosis, the first actuator moveable in conjunction with movement of the at least one of the user's knee joint. The power assisted orthosis may also include a second actuator attached to the hip orthosis, the second actuator moveable in conjunction with movement of the user's hip joints, and a valve in fluid communication with a port of at least one of the first and second actuators, where the valve controls fluid flow within the at least one of the first and second actuators to lock or unlock at least one of the first and second actuators.

### CROSS-REFERENCE TO RELATED APPLICATION

This application claims the benefit from U.S. Provisional Patent Application No. 61/829,383 entitled “Power Assisted Orthosis with Hip-Knee Synergy” filed on May 31, 2013, which is hereby incorporated in its entirety by reference.

### GOVERNMENT SPONSORSHIP

This invention was made with the assistance, at least in part, from United States government support awarded by the Department of Defense under PR043074 and W81XWH-05-1-0389 and by the Department of Veterans Affairs under A6404R and B3463R.

### FIELD OF INVENTION

The present invention generally relates to a fluid-controlled orthosis device capable of selectively bracing, dampening, and powering movement of the hip and knee joints to assist persons having weakened or paralyzed muscles with walking, ascending and descending stairs, sitting, and other leg movements.

### BACKGROUND

Functional neuromuscular stimulation (“FNS”) is a medical treatment by which low-level electrical current is applied to nerves that control muscles to stimulate functional movements.

FNS may be used to activate and strengthen persons' paralyzed or weakened leg, hip, and trunk muscles following spinal cord injury, stroke, multiple sclerosis, or brain injury.

Control of knee stability and dynamics during walking by FNS alone, however, is not always feasible because continually applying FNS to the muscles quickly causes muscle fatigue. With such persons, a controllable knee constraint mechanism presently may be used to lock and hold the knee joint in extension, without muscle or FNS use, while standing.

Walking with a conventional brace, however, is slow and exhausting because excessive effort is required to hike the pelvis, and lift and swing the leg forward when the ankle and knee joints are locked and the hips are either locked, free, or reciprocally coupled. This awkward and unintuitive form of ambulation requires five times the energy costs of normal walking at only one sixth of normal gait speed, which limits typical walking distances to less than a city block. Stair ascent and descent are extremely difficult with standard braces and require that the entire body weight be lifted or lowered by the upper extremities. Conventional braces also restrict motion of the knees and ankles and couple the hips reciprocally, thus preventing a high step with one leg.

FNS can actively propel the body forward and produce large movements of the unrestrained joints suitable for achieving a high step or level walking. However, stimulated movements of the limbs are difficult to control, and walking with FNS alone is characterized by excessive forward lean and upper extremity effort, as well as rapid muscle fatigue due to the sustained stimulated contractions required to support the body against collapse.

Therefore, there is a need for an improved device for persons with paralyzed or weakened trunk, hip, knee, and ankle muscles.

### SUMMARY

An improved orthosis device with various innovative features is presented. The device may include a knee device that uses fluid (such as hydraulic or magnetorheological fluid or inert gas), valves, and other components to lock, unlock, couple, and dampen movement in the knee joints based upon user selections, sensor feedback to a controller, or a combination of both. The knee device joint may comprise a 4-bar linkage, which is preferable to a standard hinge joint because the knee does not pivot as a hinge. Instead, the normal function of the knee during walking has both rotation and translation of the joint center. Thus, the 4-bar linkage better matches the center of rotation of an anatomical knee joint, thereby providing smoother operation of the device. Such a knee device is an improvement over prior orthoses because it better mimics natural knee movements during walking and other activities.

The device may also include a hip device that locks, unlocks, and couples movement in the hip joints based upon user selections, sensor feedback to a controller, or a combination of both. In a novel improvement, the hip device may be coupled with the knee device to achieve an intermittent reciprocal coupling of a user's hips together with a hip and knee synergy so that the knee and hip joints function together to more closely approximate normal joint movements during standard activities such as walking and stair ascending and descending.

In another improvement, a power assist function may be added to the device to provide additional joint motion when the user's muscle power (whether or not aided by FNS) is insufficient. For example, the power assist feature may be employed to provide greater hip flexion to take a step or ascend stairs.

Power assistance may be provided by a motor and pump to re-pressurize a hydraulic, pneumatic or magenetorheological circuit, or by efficient, back-drivable electrical motors at the joints incorporated into the control and actuation system that may also achieve joint coupling electronically by the control system, rather than by direct mechanical connections.

A power-assisted orthosis is shown and described. The power assisted orthosis may include a knee orthosis adapted to be secured to at least one of a user's knee joint, a hip orthosis adapted to be secured to a user's hip joints, and a first actuator attached with the knee orthosis, the first actuator moveable in conjunction with movement of the at least one of the user's knee joint. The power assisted orthosis may also include a second actuator attached to the hip orthosis, the second actuator moveable in conjunction with movement of the user's hip joints, and a valve in fluid communication with a port of at least one of the first and second actuators, where the valve controls fluid flow within the at least one of the first and second actuators to lock or unlock at least one of the first and second actuators.

A power-assisted orthosis may include a knee orthosis adapted to be secured to at least one of a user's knee joint, a hip orthosis adapted to be secured to a user's hip joints, and a first actuator attached with the knee orthosis, the first actuator moveable in conjunction with movement of the at least one of the user's knee joint. The power assisted orthosis may also include a second actuator attached to the hip orthosis, the second actuator moveable in conjunction with movement of the user's hip joints, a motor operatively coupled with the first and second actuators, a pump in operative communication with the motor, a reservoir in communication with the pump for storage of pressurized fluid, and a directional control valve in operative communication with the reservoir, where the directional control valve distributes fluid to coordinate motion of the first and second actuators and assist motion of the knee orthosis and hip orthosis.

A power-assisted orthosis may include a knee constraint adapted to be secured to at least one of a user's knee joint, and a back-drivable motor in operative communication with the knee constraint, where the back-drivable motor drives motion of the knee constraint and modulates damping of the user's knee joint during movement.

A knee orthosis may include an electrical stimulator, at least one electrode operatively coupled with the electrical stimulator and in operative communication with at least one nerve of a user, where the electrical stimulator is adapted to provide an electrical current to stimulate functional movement of a knee of the user. The knee orthosis may also include a knee constraint adapted to operatively attach to a leg of the user, where the knee constraint prevents unwanted movement of the knee of the user.

A knee orthosis may include a knee constraint adapted to be secured to a user's knee joint, an actuator attached with the knee constraint, the actuator having a rotary actuator or cylinder rod that extends and retracts in conjunction with movement of the user's knee joint, and a first valve in fluid communication with a port of the actuator, where the first valve controls fluid flow within the actuator to lock or unlock the cylinder rod or rotary actuator. The knee orthosis may also include a second valve or damper secured with the knee constraint, the damper or second valve configured to provide variable resistance to knee flexion and extension during movements of the user's knee joint.

A hip orthosis may include a hip constraint adapted to operatively fit a user's hip, a conversion device to convert rotatory motion of flexion and extension of the user's hip to a linear motion, and a sensor in operative communication with the conversion device, the sensor modulated to a plurality of independent states during a gait of a user.

A hip orthosis may include a hip constraint adapted to operatively fit a user's hip, an actuator in operative communication with the hip constraint, and a pump in operative communication with the actuator. The hip orthosis may also include a bi-directional control valve adapted to pressurize the actuator to cause movement of the actuator, where movement of the actuator moves the hip constraint in a predetermined manner moving the user's hip.

A power-assisted orthosis may include a knee constraint adapted to be secured to at least one of a user's knee joint, a hip constraint adapted to be secured to a user's hip joint, and a first actuator attached with the knee constraint, the first actuator controlling movement of the knee constraint. The power-assisted orthosis may also include a second actuator attached to the hip constraint, the second actuator controlling movement of the hip constraint, a valve in fluid communication with the first and second actuators, where the valve controls fluid flow within the first and second actuators to control movement the first and second actuators, an electrical stimulator in operative communication with at least one nerve of a user, where the electrical stimulator is adapted to provide an electrical current to stimulate functional movement of the user's knee joint, hip joint or both, and a controller operatively coupled with the electrical stimulator and the valve to lock, unlock, dampen, and/or couple movements of the user's hip and knee joints to approximate normal movements.

### BRIEF DESCRIPTION OF THE DRAWINGS

The operation of the invention may be better understood by reference to the detailed description taken in connection with the following illustrations, wherein:

FIGS. 1-A and 1-B are perspective views of the device with knee mechanism and hip mechanism;

FIG. 2 is perspective view of the knee mechanism feature of the device;

FIG. 3 is a schematic of the knee mechanism feature of the device;

FIG. 4 is a diagram of the 4-bar linkage feature at the knee of the device;

FIG. 5 is an elevation view of the 4-bar linkage feature at the knee of the device;

FIG. 6 is a table providing geometry and positioning specifications for one embodiment of the 4-bar linkage feature at the knee of the device;

FIG. 7-A is a perspective view of an embodiment of the hip mechanism feature of the device;

FIG. 7-B is a perspective view of another embodiment of the hip mechanism feature of the device;

FIG. 7-C is a perspective view of another embodiment of a hip and knee mechanism of the device that may include adjustable uprights with fixed piston mountings to preserve moment arm relationships, and a simplified three bar linkage at the hip that may eliminate the complex rack-and-pinion transmission;

FIG. 8 is a perspective view of a user wearing the hip mechanism feature of the device;

FIG. 9-A is a schematic of the hip mechanism feature of the device in a first operating state;

FIG. 9-B is a schematic of the hip mechanism feature of the device in a second operating state;

FIG. 10 is a schematic of the device with optional power-assist function and the hip-knee coupling;

FIG. 11 is a diagram showing valve positioning in the device for each phase in a user's stair ascent for an embodiment;

FIG. 12 is a perspective view of the knee damper feature of the device;

FIG. 13 is a diagram showing valve positioning in the device with a damper feature for each phase in a user's stair descent for an embodiment;

FIG. 14 is a diagram showing controller function for an embodiment of the device;

FIG. 15 is a diagram showing controller function during stair descent for an embodiment of the device;

FIG. 16 is an elevation view of the device with the knee and hip cylinder oriented along the user's thigh;

FIG. 17 is a perspective view of a portion of the device utilizing rotary actuators; and

FIG. 18 is a perspective view of a rotary actuator used with the device.

FIG. 19 is a perspective view of the rotary hydraulic actuator.

FIG. 20 is an exploded view of the rotary actuator of FIG. 18.

FIG. 21 is a perspective view of the rotary actuator with an integrated valve.

FIG. 22 is a perspective view of the rotary actuator with integrated valve with a portion thereof in phantom.

FIG. 23 is a perspective view of the rotary actuator with integrated valve with a portion thereof in phantom.

### DETAILED DESCRIPTION

Reference will now be made in detail to exemplary embodiments of the present invention, examples of which are illustrated in the accompanying drawings. It is to be understood that other embodiments may be utilized and structural and functional changes may be made without departing from the respective scope of the invention. Thus, by way of example, when this specification describes a user with paralyzed leg muscles walking with the aid of FNS, it is to be understood that such description is provided as a non-limiting example and that the invention described herein may also be useful for persons having weakened muscles moving their limbs under their own muscular power or with the assistance of FNS or by some other means. Moreover, features of the various embodiments may be combined or altered without departing from the scope of the invention. As such, the following description is presented by way of illustration only and should not limit in any way the various alternatives and modifications that may be made to the illustrated embodiments and still be within the spirit and scope of the invention.

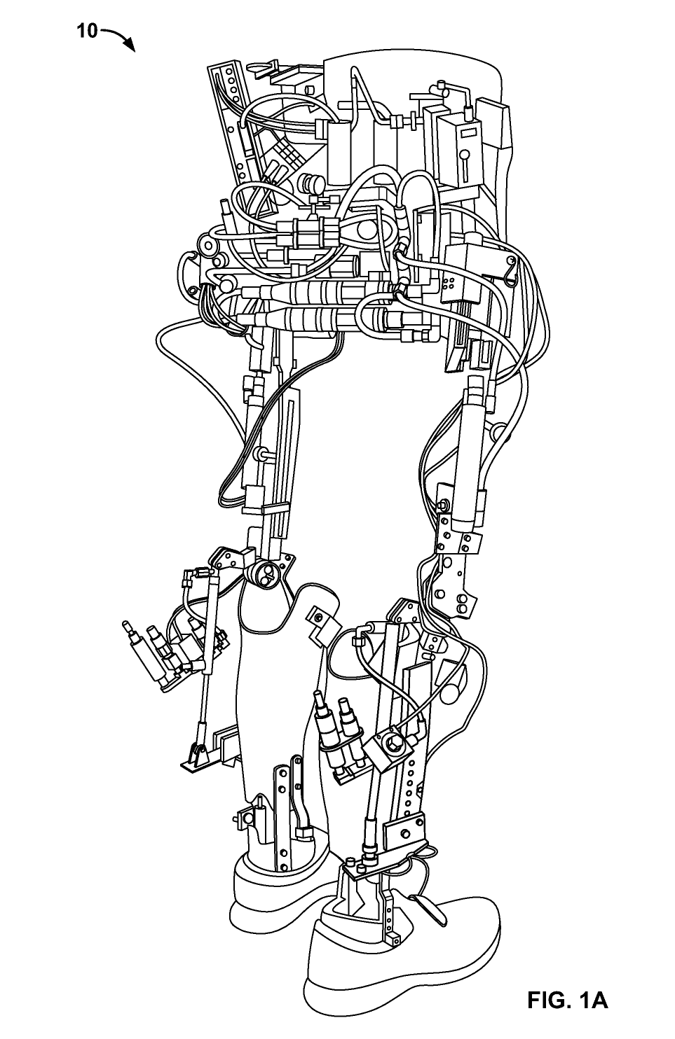

A device 10 is generally provided, as illustrated in FIGS. 1-A and 1-B.

### Knee Mechanism

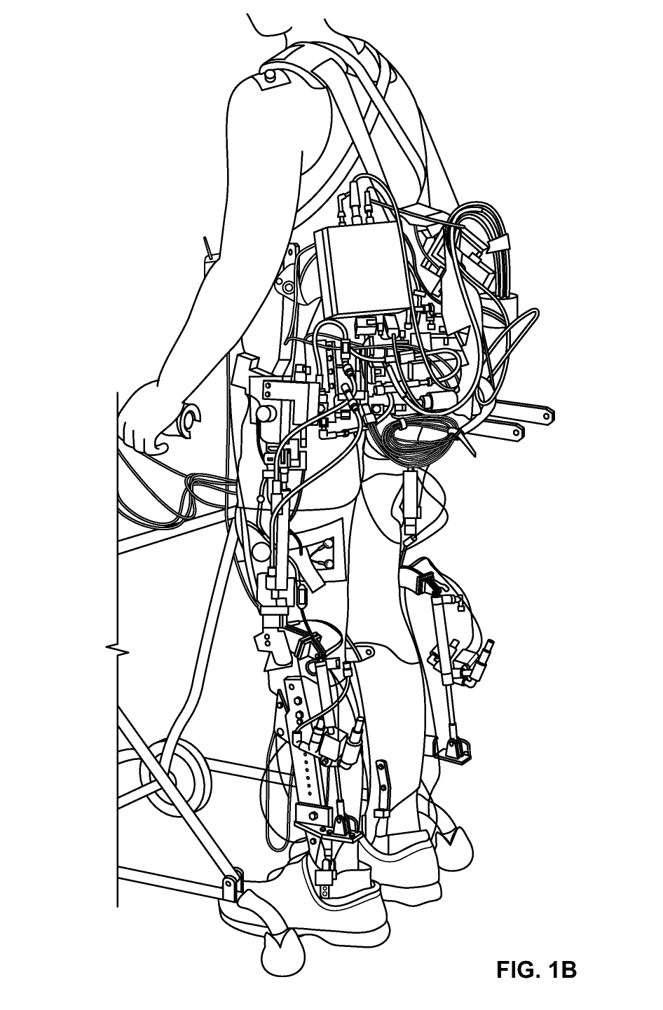

As shown in FIG. 2, device 10 may include a knee mechanism 20 attached to a user's leg by straps or by other suitable means at locations on the thigh, on the lower leg, and in other locations as appropriate to secure mechanism 20 to the patient's leg. Mechanism 20 may be attached to one or both of the user's legs. Mechanism 20 may be adjustable to fit a variety of users with different size legs.

Mechanism 20 may include a lower bar 21 that may connect to an upper bar 22 at a knee joint 23. In the embodiment shown in FIG. 2, knee joint 23 is a pin joint, although knee joint 23 may be another type of joint, such as rack and pinion or a 4-bar linkage as described later in this specification.

Lower bar 21 may include a lower bracket 24 that may be attached to lower bar 21 by a carriage bolt inserted through aligned apertures of lower bar 21 and lower bracket 24 and secured by a nut, or by other suitable means for attachment. Lower bracket 24 also may be attached to a cylinder 25. As shown in FIG. 2, cylinder 25 may comprise a clevis 27 affixed or integral with on one or both longitudinal ends of cylinder 25. Cylinder 25 may also include a cylinder rod 26 that extends and retracts within the body of cylinder 25. By way of a non-limiting example, cylinder 25 may function hydraulically, pneumatically, or by some other fluid, such as magnetorheological fluid or gas. As shown in FIG. 2, cylinder 25 may comprise a double-acting hydraulic cylinder. In one embodiment, cylinder 25 may be attached to lower bracket 24 and an upper bracket 29. As illustrated in FIG. 2, lower bracket 24 may be attached to cylinder rod 26 by clevis 27, and upper bracket 29 may be attached to cylinder 25 by clevises 27.

In the embodiment shown in FIG. 2, when knee joint 23 pivots so that the angle between upper bar 22 and lower bar 21 increases, the rod 26 of cylinder 25 extends. That movement corresponds with extension of the user's knee. Conversely, when knee joint 23 pivots so that the angle between the two bars decreases, rod 26 retracts. That movement corresponds with flexion of the user's knee.

Upper bracket 29 may be attached to upper bar 22 in the same manner as lower bracket 24 and lower bar 21 are attached or by other suitable means.

Upper bracket 29 and lower bracket 24 may have adjustable attachments to upper bar 22 and lower bar 21, respectively. For example, in one embodiment, lower bar 21 and upper bar 22 may have a plurality of apertures along their lengths. A bolt, pin, or other fastening device, may be inserted through different aligned apertures of the lower bracket 24 and lower bar 21 and of upper bracket 29 and upper bar 22, depending on the length of cylinder 25 or other factors.

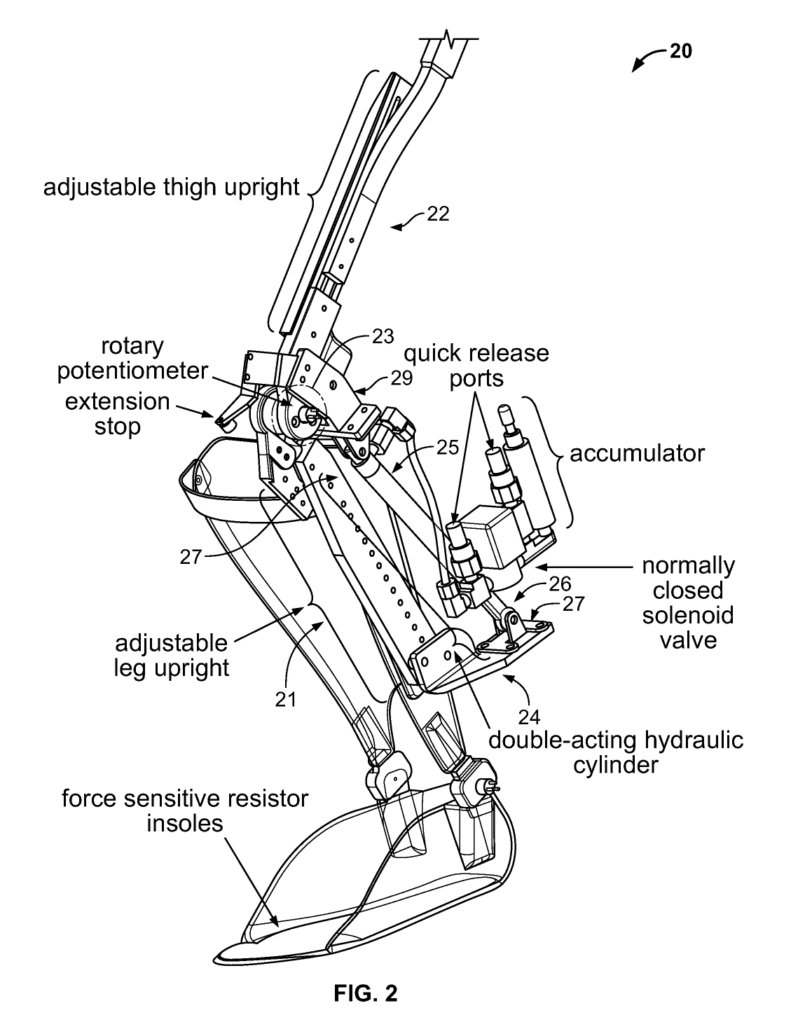

A schematic of one of the embodiments is shown in FIG. 3. As shown, flexion of the user's knee will act to retract cylinder rod 26, while knee extension will act to extend cylinder rod 26.

A valve 201 may be employed. Valve 201 may comprise a normally closed, 2-way, 2-position valve. Valve 201 may be actuated by solenoid, manually, or by other suitable means. Valve 201 may include a port A coupled to a conduit 203 leading to a chamber within the blind side of cylinder 25. Valve 201 may also include a port B coupled to a conduit 204 leading to a chamber on the rod side of cylinder 25. As shown in FIG. 3, valve 201 is biased to a closed position. In the closed position, a poppet 202 in a seat 205 prevents fluid from moving from port B to port A.

In the closed position, cylinder rod 26 is locked against retraction because poppet 202 prevents fluid from moving out of the blind side of cylinder 25. Therefore, valve 201's normal and unpowered state locks the user's knee joint against flexion. When cylinder rod 26 has fully extended, the user's leg is locked in an extended state. That allows the user to place weight on the leg without having to use muscle power (whether powered by FNS or otherwise) to stay upright or prevent collapse.

Valve 201 may be actuated by energization of the solenoid or manually. When actuated, valve 201 moves to an open position creating an open path through valve 201 allowing fluid to flow freely in both directions between ports A and B. Thus, when valve 201 is actuated the user may freely flex or extend her knee joint.

Valve 201 also may have a safety feature allowing the user to extend her knee joint while valve 201 is in the closed position. As shown in FIG. 3, Poppet 202 may be spring-loaded to keep it pressed into seat 205. The amount of force that the spring exerts upon poppet 202 corresponds to a cracking pressure, where fluid will not flow from port A to port B unless the pressure at port A exceeds the cracking pressure needed to move the poppet 202 off of seat 205. Starting with cylinder rod 26 fully retracted (corresponding to a knee in the flexed position), as the user begins to extend her knee (whether under her own power, by FNS, or by some other means), the fluid pressure at the rod side of the chamber within cylinder 25 and at port A increases. When the pressure at port A reaches the cracking pressure of poppet 202, fluid will flow into the blind side of the chamber within cylinder 25, allowing cylinder rod 26 to extend and the user to thereby extend her knee joint. The cracking pressure of check valve 202 may be set at a pressure that the user has sufficient muscular strength (aided by FNS or otherwise) to overcome. Thus, if valve 201 is a solenoid and if power is lost, the user may still extend her knee joint while valve 201 is in the closed position by extending with enough force to overcome the cracking pressure.

As the user extends his knee, poppet 202 prevents fluid from flowing out of the blind side of the cylinder chamber, thereby locking against knee flexion. When cylinder rod 26 has fully extended, the user's leg is locked in an extended state. That allows the user to place weight on the leg without having to use muscle power (whether powered by FNS or otherwise) to stay upright.

As shown in FIG. 3, an accumulator 203 may be connected to conduit 204 to take up the fluid volume differential between the blind and rod sides of cylinder 25. Alternatively, other means known in the art may be used to take up the volume differential between cylinder sides in a hydraulic system, such as introducing air bubbles into the system or using expandable hosing as conduit.

Further, the knee mechanism 20 may include a force sensitive resistor insole 28. The force sensitive resistor insole 28 may be pivotally attached with the lower bar 22. In some embodiments, a second bar or rod 32 may be pivotally attached with the force sensitive resistor insole 28 and may be operatively secured with the lower bar 21 in any appropriate manner. The force sensitive resistor insole 28 may provide feedback to the knee mechanism 20 (or a computer as described below) as to the force being applied to the foot of the wearer.

### 4-Bar Linkage

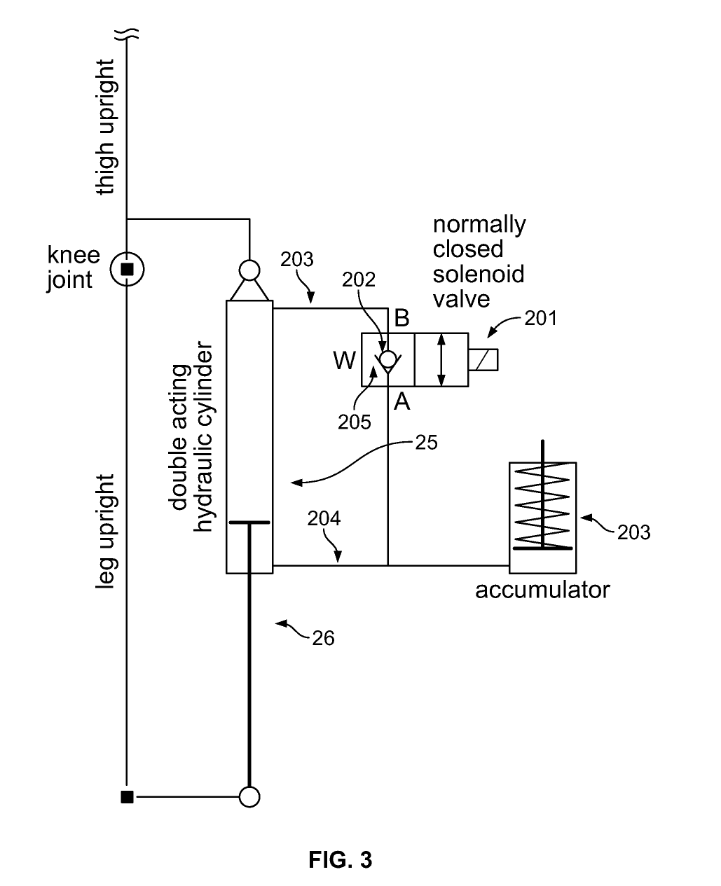

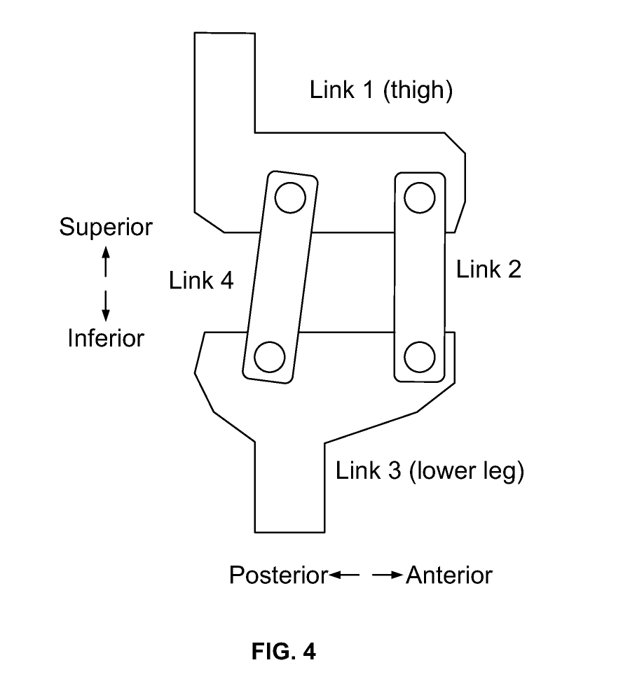

As shown in FIGS. 4 and 5, knee joint 23 may be a 4-bar linkage connection. A human knee joint is not purely rotational, but rather includes some translation in the anterior-posterior direction. Thus, the instantaneous center of rotation (the “ICOR”) between the thigh and lower leg segments changes while the knee extends and flexes. Thus, a 4-bar linkage may better mimic the rotation of a human knee joint than a standard hinge joint.

The ICOR of the 4-bar linkage may be configured to closely align with the trajectory of the anatomical knee center during the full range of motion to create a comfortable fit and to prevent binding. The linkage may be mounted to adjustable thigh and leg uprights to align the linkage with the user's knee center at full extension.

As shown in FIG. 4, link 1 may be part of (or rigidly affixed to) the thigh upright of the device. Link 3 may be part of (or rigidly affixed to) the lower leg upright of the device. Link 1 may connect the superior revolute joints of links 2 and 4. The anterior rocker (link 2) may be connected by revolute joints to link 1 and the coupler (link 3). The coupler may connect the anterior rocker (link 2) to the posterior rocker (link 4) by two revolute joints.

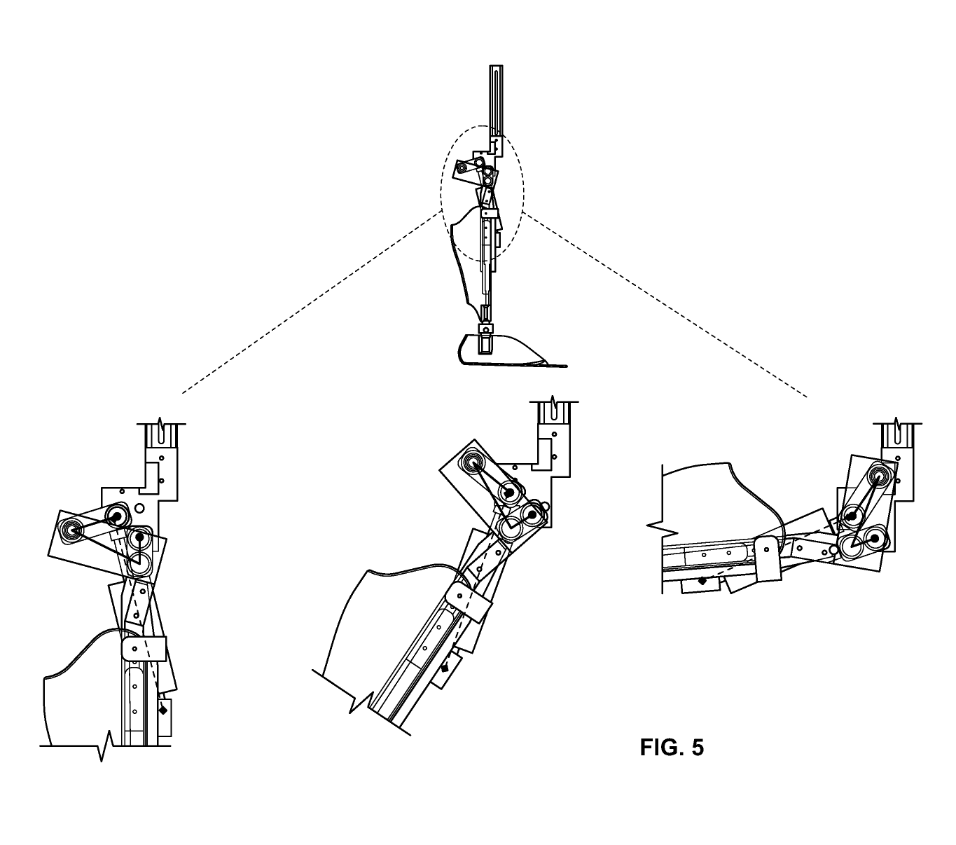

By way of non-limiting example, FIG. 6 shows one of the embodiment's bar lengths and cylinder mounting (clevis) positions in the sagittal plane relative to anatomical knee center at full extension. Coordinates (x,y) correspond to (anterior(+)-posterior(−), superior(+)-inferior(−)) directions.

Links 2 and 4 may be bars constructed of aluminum or other suitable material. Links 1 and 3 may be plates constructed of aluminum or other suitable material. Links 2 and 4 may be attached to links 1 and 3 by shoulder bolts or other suitable means. The revolute joints may be comprised of nylon flanged bearings around the shafts, and thrust needle-roller style bearings may be placed on either side of the link to smooth rotation and minimize wear.

The knee mechanism 20 may further include an extension stop 34 secured with the knee joint 23 in any appropriate manner. The extension stop 34 may generally prevent over extension of the knee mechanism 20 or more specifically the knee joint 23 passed a predetermined amount of rotation.

The knee mechanism 20 may include a rotary potentiometer 35 operatively coupled with the knee joint 23. The rotary potentiometer 35 may be used as a sensor to determine the amount of rotation of the knee mechanism 20 or more specifically the knee joint 23. This information may be utilized to operate the knee mechanism 20 and/or to provide feedback to the patient or clinician.

### Hip Device

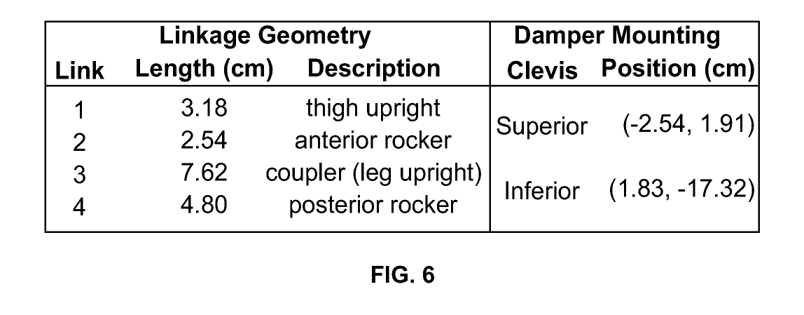

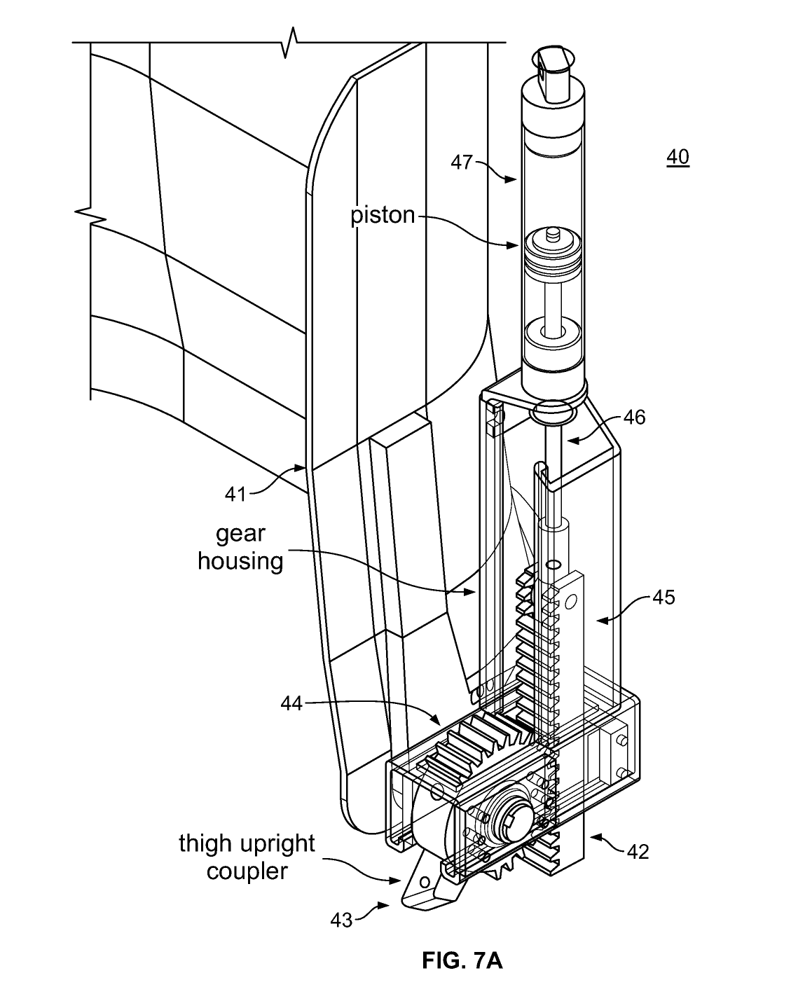

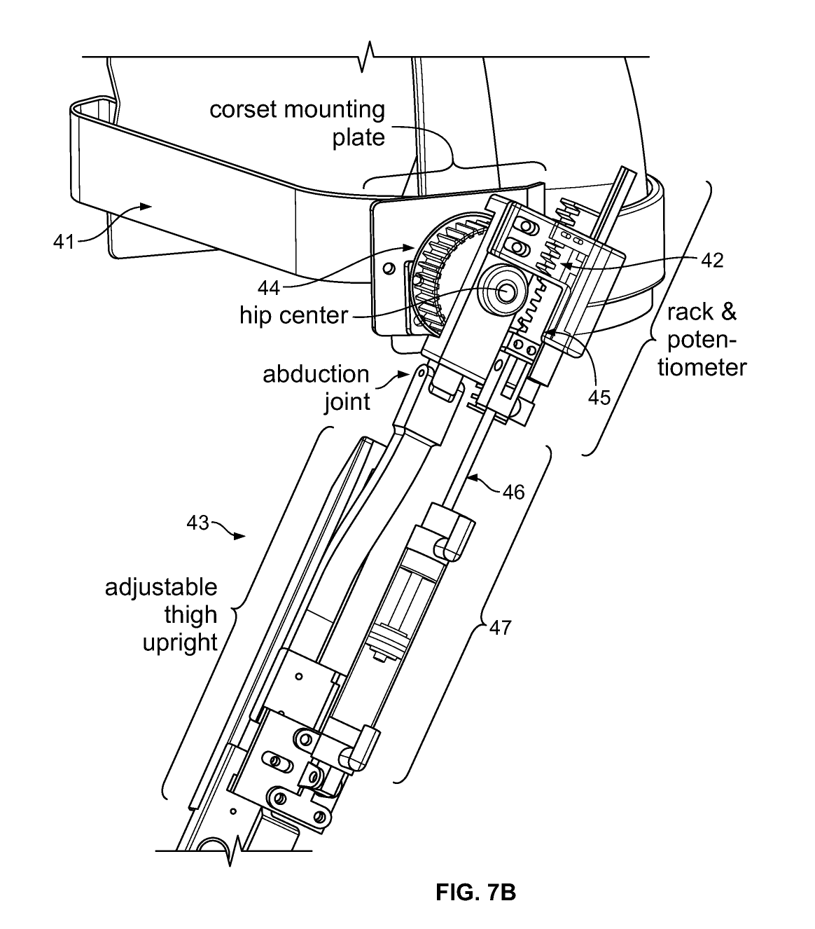

As shown in FIGS. 7-A and 7-B, device 10 may include a hip mechanism 40. The hip mechanism 40 may be attached at the user's hip by means of a corset 41. The hip mechanism may include a conversion device 42 for converting the rotary motion of the flexion and extension of the user's hip joint to linear motion (cylinder extension/retraction).

In the embodiment shown in FIGS. 7-A and 7-B, conversion device 42 may be a rack-and-pinion arrangement in which a thigh bar 43 is attached by a strap or other suitable means to the user's thigh. As shown, thigh bar 43 may be welded or otherwise attached to a geared pinion 44, such that flexion of the user's hip rotates the pinion 44 in a direction that causes a rack 45 to move in a downward direction. Thus, in the embodiment shown in FIG. 7-A, hip flexion corresponds with the extension of a cylinder rod 46 within a cylinder 47. In the embodiment shown in FIG. 7-B, hip flexion corresponds with the retraction of cylinder rod 46 within cylinder 47. It is to be understood that conversion device 42 may be any device known in the art that converts rotary to linear motion (or linear to rotary motion) that is suitable for the application. By way of a non-limiting example, cylinder 47 may function hydraulically, pneumatically, or by some other fluid such as magnetorheological fluid.

Conversely, in the embodiment shown in FIGS. 7-A and 7-B, hip extension would rotate pinion 44 in the opposite direction and cause rack 45 to move in an upward direction. Thus, in the embodiment shown in FIG. 7-A, hip extension corresponds to the retraction of cylinder rod 46. And in the embodiment shown in FIG. 7-B, hip extension corresponds to extension of cylinder rod 46.

In other embodiments of the device, the rack and pinion 44 depicted in FIGS. 7-A and 7-B may be replaced by a three-bar linkage such as that shown in 7-C. This may reduce weight, complexity and facilitate fabrication and maintenance of the device. Also depicted in FIG. 7-C are alternative configurations of thigh and shank uprights in which the hip and knee actuating cylinders may be located centrally to the structure, rather than lateral or posterior as shown in FIGS. 7-A and 7-B. This configuration may generally avoid deforming bending torques in the frontal plane to enhance reliability and robust operation.

The structure in 7-C may also maintain fixed relationships between the pistons and their connection points by sliding the piston mountings within an exterior structure for rapid adjustment of segment lengths for users of various sizes without altering the moment arms of the cylinder and joint arrangements.

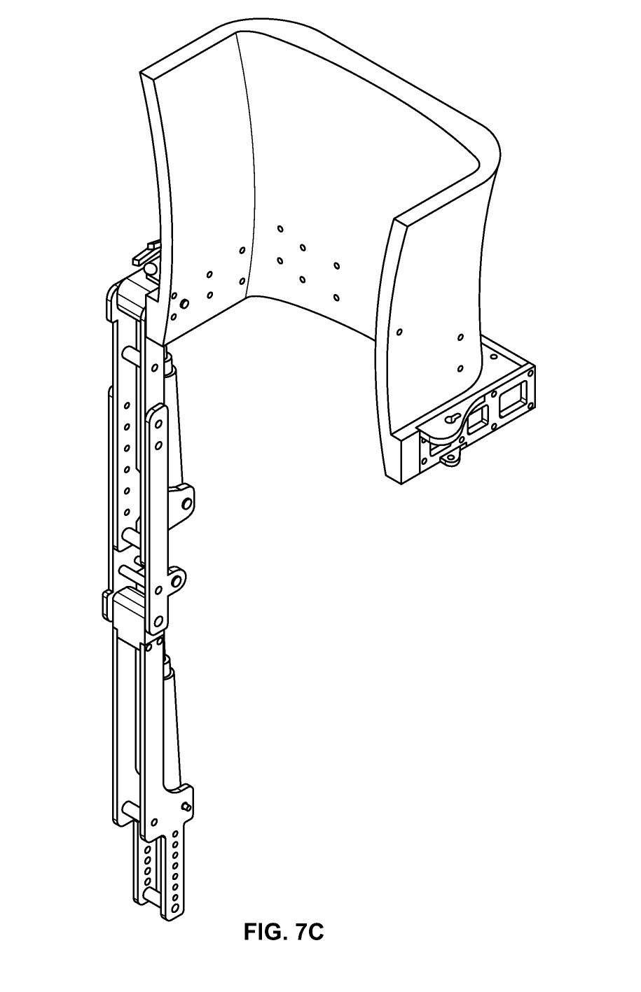

As shown in FIG. 8, hip mechanism 40 may comprise a conversion device 42 attached at each of the user's hip joints. Hip mechanism 40 may be configured so that each conversion device 42 may be modulated independently among four states during gait: (1) reciprocally coupled; (2) freed; (3) bi-directionally locked; and (4) unidirectionally locked.

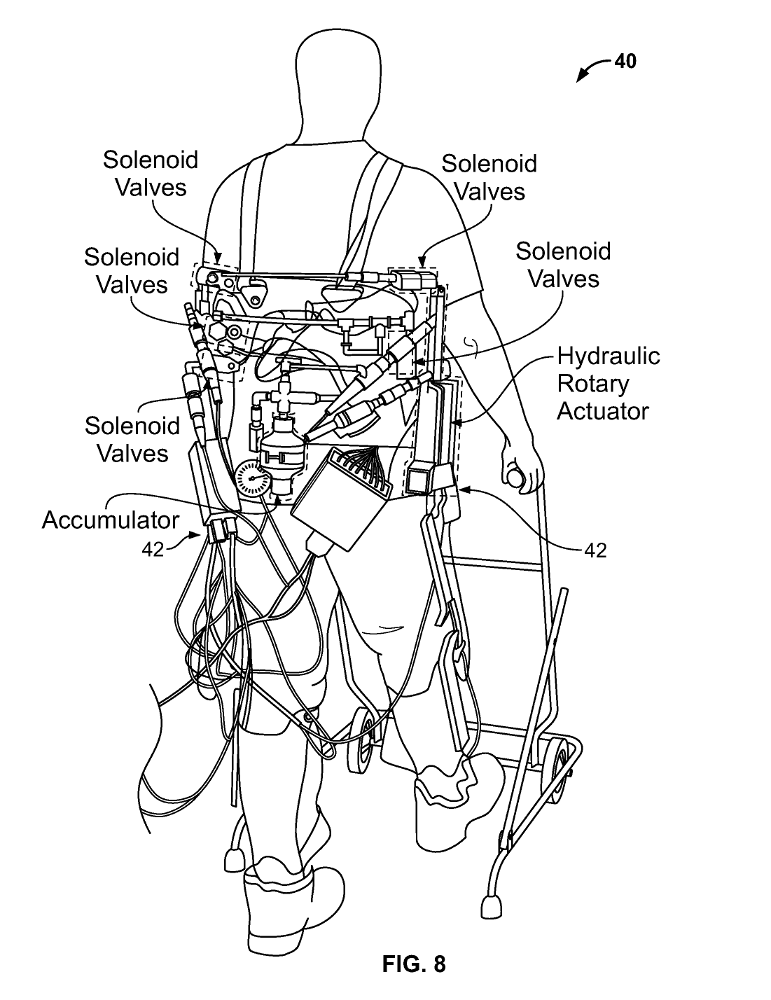

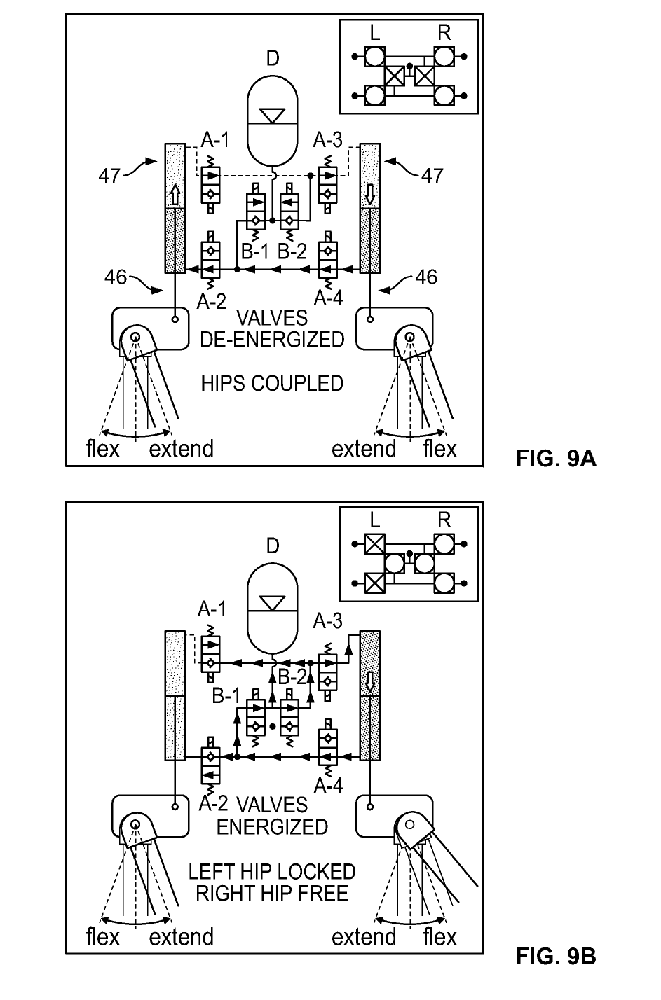

A schematic of one of the embodiments of hip mechanism 40 is shown in FIG. 9. As shown, a two-way, two-position, normally open (NO; open to flow when de-energized) solenoid valves A may be positioned at each port of the cylinders 47. Two 2-way, 2-position, normally closed (NC; closed to flow when de-energized) valves B may be included in the system to modulate the flow of fluid between the blind and rod ends of the hydraulic circuit and into an accumulator D.

As shown if FIG. 9-A, cylinder rods 46 may be reciprocally coupled when all of the valves are in their normal (or de-energized) state. In that state, when one cylinder rod is extended by the flexion of the user's hip joint, fluid flows into the rod end of the opposite cylinder 47 to cause cylinder rod 46 to retract, thereby causing the user's opposite hip joint to extend.

By actuating specific valves, the cylinder rods may be individually locked or freed to move in one or both directions. By way of a non-limiting example, as shown in FIG. 9-B, a cylinder rod may be bi-directionally locked by actuating valves A-1 and A-2, thereby preventing flow from either end of the cylinder. Similarly, the opposite cylinder rod may be bi-directionally locked by actuating valves A-3 and A-4.

Bi-directional free piston motion in the right hip cylinder may be achieved by actuating valves A-1, A-2, B-1, and B-2 as shown in FIG. 9-B. In that state, fluid may flow freely from one end of the cylinder to the other end of the same cylinder and into an accumulator D, which takes up the fluid volume differential between the cylinder blind and rod sides.

Unidirectional piston locking may be achieved by activating the NO valve at the rod end of either cylinder (A-2 or A-4) to lock against extension of cylinder rod 46 and activating valve B-2. Similarly, cylinder rod 46 may be locked against retraction by activating the NO valve at the blind end (A-1 or A-3) and activating valve B-1. Thus, the rod end NO and blind end NC valves would be actuated to prevent piston extension and allow piston retraction and the blind end NO and rod end NC valves would be actuated to prevent piston retraction and allow piston extension. However, when cylinder rod 46 is moved in the direction of the free cylinder end, vacuum formation in the locked cylinder end may resist the free motion. To avoid this effect, a feed back signal may be employed, such as a force applied to the rod-side or blind-side pressure, to deactivate the closed NO valve.

### Hip-Knee Synergy

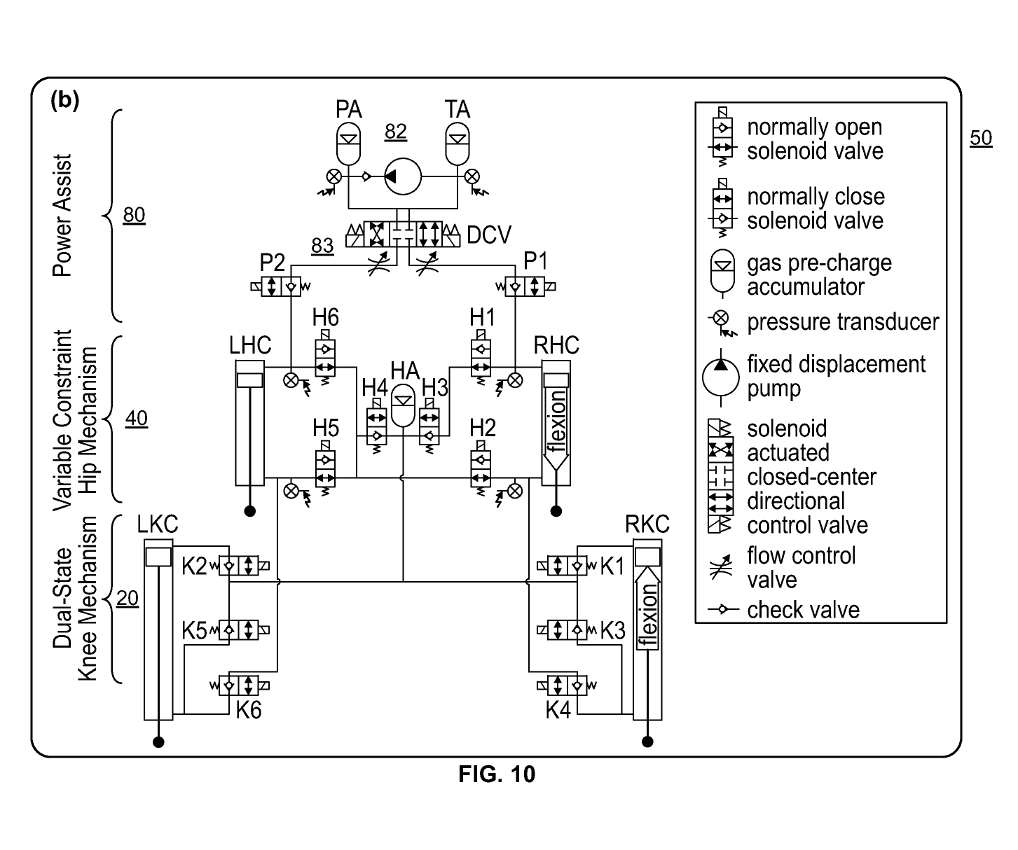

In yet another embodiment, a hip-knee coupled system 50, which is illustrated schematically in FIG. 10, may be employed. In coupled system 50, device 10 may have the fluid system used in hip mechanism 40 connected to the fluid system in knee restraint 20. The fluid may move through the system through an appropriate conduit, including (without limitation) tubing, flexible hosing, or rigid piping. Connecting the two systems improves coupling and synchronizing of hip and knee movement to better mimic a normal human gait.

By way of non-limiting example, to achieve a high step with the right leg, the system shown in FIG. 10 would couple right side knee and hip flexion by closing valve H2 and opening valve K4 to connect the rod side of the right hip cylinder (RHC) with the rod side of the right knee cylinder (RKC). Opening valve K1 provides a flow path for extra fluid from the blind side of the RKC to the hip accumulator (HA). Right hip flexion would cause the RHC to extend and displace fluid into the rod side of the RKC, forcing it to retract. Such action would push fluid from the blind side of the RKC into HA. The force from the retraction of the RKC would be converted into a torque which would cause the user's knee to flex as a function of the change in hip angle, RHC and RKC rod side piston area ratio, and linear-to-rotary transmission ratios of the hip and knee mechanisms.

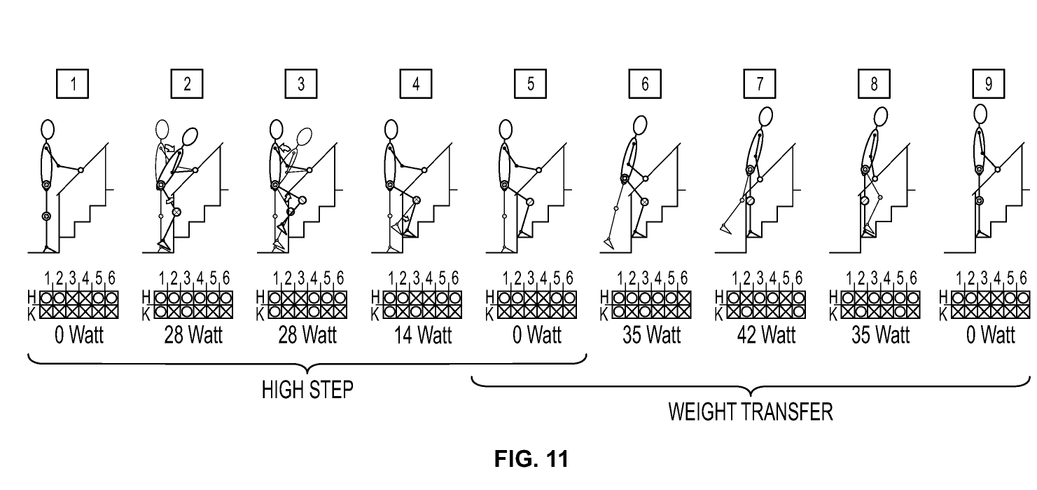

Stair ascent, which is best shown in FIG. 11, with the coupled system may consist of two basic maneuvers: (1) a high step; and (2) weight transfer onto the leading limb. The coupled system may include hip and knee angle sensors, pressure transducers at each cylinder port, and force sensitive resistors (FSR) embedded in the soles of each shoe to provide feedback signals to control the valves for context-dependent coupling during stair ascent

As an example, shown in FIG. 11, the high step maneuver may be separated into any number of discrete phases. Below each phase, FIG. 11 provides a chart showing whether each valve in hip-knee coupling mechanism in FIG. 10 is open (“o”) or closed (“x”) in that particular phase. Below the chart, FIG. 11 provides the estimated instantaneous power required to power the solenoid valves.

In Phase 1, the hips are coupled and the knees are locked to provide stable standing. No valves need to be actuated in Phase 1.

Phase 2 may be initiated by the user operating a manual switch, by a control system signal, or by other suitable means. In Phase 2, both hips are freed to allow the trunk to tilt forward under the control of the upper extremities. Ipsilateral hip flexion may be initiated by FNS (or by the user's own muscular power) and the ipsilateral knee may be unlocked by energizing valves K1 and K3.

Once maximal hip and knee flexion have been achieved as determined by the joint angle sensors, Phase 3 begins by reciprocally coupling the hips by closing valve H3, and ipsilateral knee and hip flexion are coupled by closing valve K3 and opening valves K1 and K4. If FNS (or the user's muscle power or other means of power assist) alone is insufficient to clear the step, the user can push up on the railing to further extend the contralateral hip, and take advantage of the reciprocal coupling of the hip mechanism to improve ipsilateral hip and knee flexion through the coupled system 50.

In Phase 4, the knee is uncoupled from the hip and the foot is positioned over the step. Force sensing resistors, or other contact sensors, may be attached to the bottom of the user's foot to detect foot contact in Phase 5, and the knee would then be locked to stabilize the limb.

The weight transfer maneuver for ascent begins with Phase 6 by the user operating a manual switch, by a control system signal, or by other suitable means. The ipsilateral joints are unconstrained to allow ascent by stimulation to the hip and knee extensors, supplemented by the upper extremities or power assist as needed. Additional power for ascent may come from stimulation of trunk and ankle extensors or by other means of applying supplemental power.

With the knee fully extended (Phase 7), device 10 locks the hip and knee of the leading leg against flexion while the hip and knee of the trailing limb are coupled and flexed with FNS (or the user's muscle power) to clear the lip of the upper step. The ankle may be locked, controlled volitionally, by FNS, or by other means of assistance.

Phase 8 begins with the trailing hip and knee unconstrained as the leg is positioned over the step with FNS (or the user's muscle power or power assist) and extends into stance.

The hip mechanism is re-coupled reciprocally and both knee joints of the exoskeleton are locked in Phase 9. No power to the exoskeleton or FNS is required in this phase, which allows the user to rest until the sequence of maneuvers to ascend the next step is initiated.

### Power Assist

In yet another embodiment shown in FIG. 10, a power assist system (“PAS”) 80 may be provided when the user's hip muscles (whether or not assisted with FNS) are insufficient to generate the hip flexion needed to, for example, take a step or ascend stairs.

As shown, PAS 80 may utilize a motor, pump 82, and directional control valve 83 to pressurize fluid in an accumulator and distribute it to the pistons of the hip mechanism as needed for the user to obtain the necessary hip flexion.

More specifically, as shown in FIG. 10, PAS 80 may include a pump 82 and a 4-way, 3-position directional control valve (DCV) 83 positioned between a pressurized accumulator (PA) and a second tank accumulator (TA) charged to system resting pressure. When additional torque is required to flex the right hip, valve H1 may first be closed to isolate the blind side of the right hip cylinder from the rest of hip mechanism 40. The blind side of the RHC is exposed to the pressure stored in PA by opening valve P1 and shifting the position of the DCV to the right. Flow from PA pressurizes the blind side of the RHC, which extends the cylinder and applies a flexion torque to the right hip joint.

This extra torque may also be employed to assist left hip extension by reciprocally coupling the hips, or to flex the right knee via the hip/knee synergy achieved by hip-knee device 10. Once the supplemental torque is no longer needed, the DCV 83 may be de-energized and shifted to the closed-center position, P1 is closed, and H1 is reopened.

A similar sequence may supply supplemental flexion torque to the left hip. The pump is then activated to transfer fluid from TA to PA and recharge PA to its initial pressure. This reduces the pressure of TA below system resting pressure. P1 is opened and the DCV is shifted left, opening hip mechanism 40 to TA. Because P2 is closed, PA is isolated from the hip mechanism and does not pressurize the system. Instead an equal amount of fluid displaced during the power assist period flows into TA, causing TA and the hip mechanism to return to steady state. Once hip mechanism 40 system pressure has been restored, P1 and the DCV are closed and the system operates exactly as if the PAS 80 were never activated.

There are several advantages of using flow from an accumulator rather than directly from a pump to displace the cylinder pistons. First, pressure from PA may be applied to the cylinder immediately after the DCV has opened, resulting in a rapid increase in torque. Thus, the pump may not need to generate pressure rapidly and a simple fixed displacement pump may be used instead of a larger pressure-compensated variable displacement design. Second, movements of the hydraulic pistons and rotary actuators may be easily modulated by the flow control valves without the difficulties associated with precisely controlling the motor and pump. Third, pump activation may be isolated from valve activation, reducing the instantaneous power requirements of the system. Finally, safety is enhanced because the pump is isolated from the user and does not attempt to actively maintain pressure in the event of a leak.

The pump and other components requiring electrical power (such as solenoid valves) may receive power from one or more batteries carried by the user or by other suitable power sources.

In another embodiment, the pump, motor and directional control valves may be replaced with small, low-speed, high torque, low profile motors attached to back-drivable transmissions such as harmonic (strain wave gearbox) or elliptical drives at each hip joint. These embodiments may beneficial over other non-hydraulic powered exoskeletons in that this configuration may be rotated easily even when unpowered, offering little resistance to movement which may be advantage for users of FNS or with weak voluntary muscles. Hip-to-hip coupling may still be achieved hydraulically as depicted in FIG. 10, or entirely electronically by the control system rather than physical connections between the joints. This may also provide the capability of continuously varying the hip flexion-to-contralateral-extension ratio, rather than having it set to a single value determined by the fluid volume of the pistons.

The motor and back-drivable transmission option at the hips may have the additional advantage of allowing the hip joints and uprights to be disconnected from the mounting jacket to facilitate donning, doffing and storage of the device. The motors may assist FNS driven or weak voluntary movement, power the movement entirely, or achieve the same functionality of the hydraulic variable coupling hip mechanism by locking, unlocking or reciprocally coupling the hip joints electronically without the hydraulic circuit.

### Damper

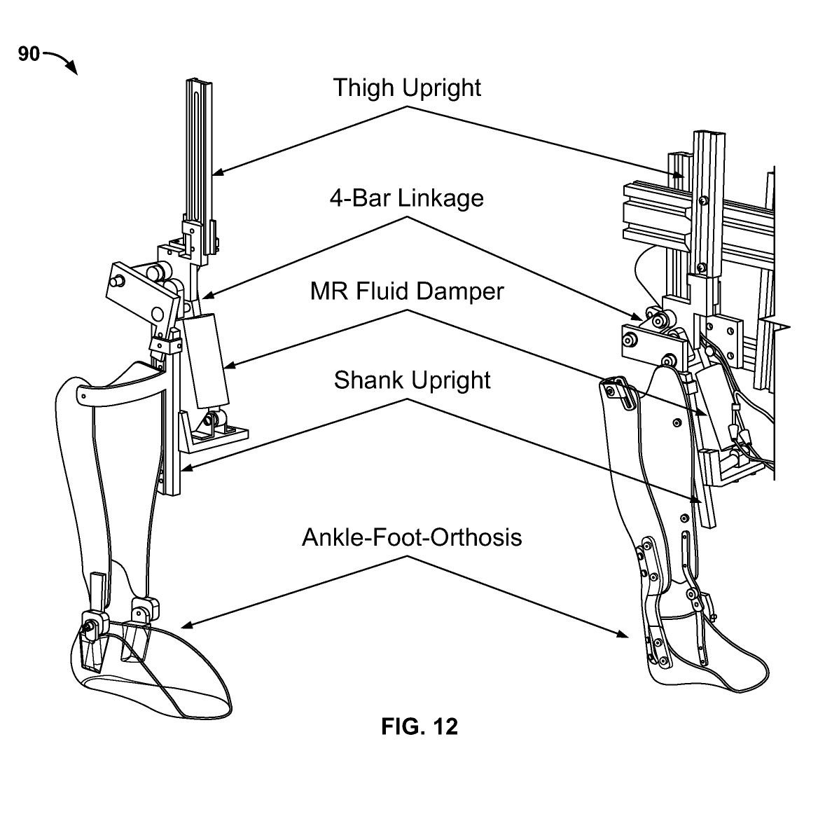

In another embodiment, as illustrated in FIG. 12, device 10 may include a damper 90 to provide variable resistance to knee flexion and extension during movements, such as stair descent, where the user desires a variable resistance force to flexion, as opposed to merely a free or completely locked joint. By way of non-limiting example, for weight acceptance during stair descent or gait, a user may prefer a large change in velocity with moderate damping to absorb the initial ground impact of the leading limb. A large damping force may also simulate the eccentric contractions of the quadriceps for controlled lowering, during which relatively slow trailing leg knee flexion is necessary to descend to the lower step.

As shown in FIG. 12, damper 90 may include a magnetorheological (MR) fluid linear damper. MR fluids may consist of micron sized ferrous particles (20-40% of the fluid by volume) suspended in a carrier liquid, usually water or oil, and additives to reduce evaporation and settling. In the absence of a magnetic field (the off-state), MR fluid has Newtonian behavior with properties similar to the carrier liquid. When a magnetic field is applied the ferrous particles in the fluid align with the magnetic flux lines to resist flow, creating an offset yield stress resulting in non-Newtonian fluid behavior during the on-state. The magnitude of the offset yield stress is proportional to the magnetic field, thus variable resistance to flow can be obtained via modulation of the magnetic field intensity.

A variable damper may be created by employing MR fluid as the working medium in a hydraulic cylinder containing one or more electromagnetic coils such that the magnetic field across the fluid in the cylinder can be modulated by changing the current input to the coil.

As shown in FIG. 12, the MR damper may substitute for cylinder 25 on knee device 20, be arranged in the same manner as cylinder 25, and function the same as cylinder 25 except for the variable damping feature during the MR damper rod's extension and retraction. Alternatively, the MR damper may be added as an additional element of knee device 20, to operate in parallel with cylinder 25, and to be activated when the user desires (or the control system triggers) a variable damping effect.

In yet another embodiment, damper 90 may comprise the schematic shown in FIG. 3 utilizing a non-MR hydraulic fluid, where valve 201 is a valve—such as a high speed or proportional valve—adapted to vary flow based on an adjustable current supplied by a pulse width modulation (a “PWM”) or by other suitable means known in the art. Adjusting the hydraulic flows and pressures within cylinder 25 achieves a hydraulic dampening effect similar to that achieved by the MR damper.

In other embodiments to achieve knee damping, a motor and transmission may be located in tandem with a simple hydraulic piston and valve. The valve and piston arrangement may form a simple linear damper for a fixed, continuous level of damping, with variable damping tuned for each intended task provided by the motor which may add resistive torque to the hydraulic baseline under software control. Similarly, a motor and transmission may be configured to continuously vary the coupling ratio between the knee and ipsilateral hip, rather than have the amount of knee flexion during coupling determined entirely by the relative volumes of the hip and knee cylinders. The motor and transmission embodiment may preserve most all, if not all, functionality at the knee joints and still allow them to be locked, free, damped or coupled to hip motion, and even provide knee extension/flexion power assist, whether they are integrated into the hydraulic circuit or not.

### Stair Descent

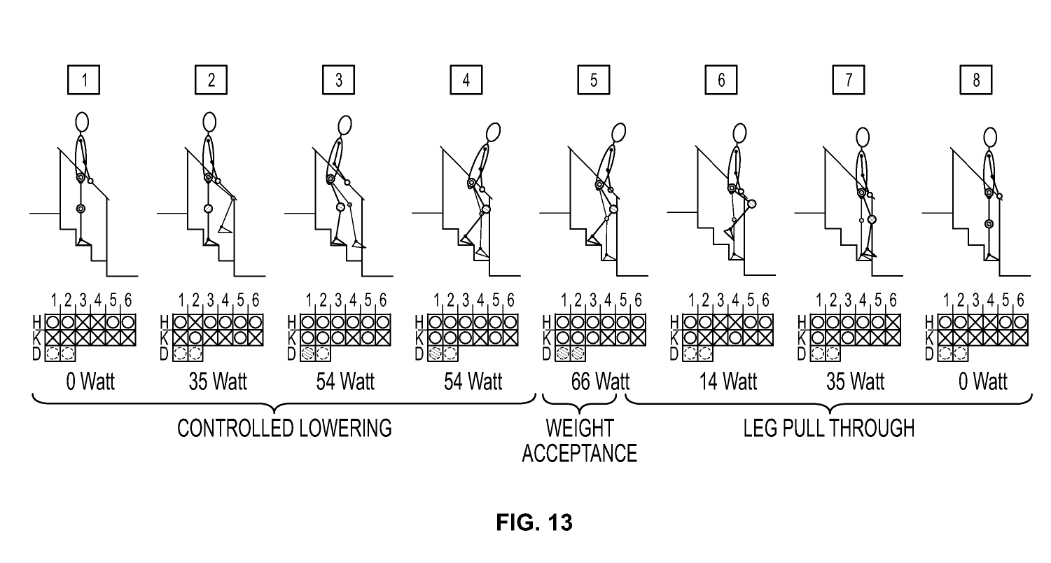

FIG. 13 illustrates how device 10 with cylinder 25 and an MR damper in parallel may function during stair descent by the user. The step-by-step descent may be characterized by controlled lowering and weight acceptance dominated by energy absorption at the knee. Stair descent with device 10 may be described in three stages: (1) controlled lowering of the body; (2) weight acceptance on the step below; and (3) pull-through of the trailing leg. The same sensor set described above with regard to stair ascent may be used for feedback control of the valves and dampers during stair descent. Below each phase in FIG. 13 a chart shows whether each valve in the circuit shown in FIG. 10 is open (“o”) or closed (“x”) in that particular phase and whether the damper is on (solid circle) or off (empty circle). In the chart, D1 refers to the damper on the user's right leg, and D2 refers to the damper on the user's left leg. Below the chart, FIG. 13 provides the estimated instantaneous power required.

As an example, controlled lowering may be subdivided into four phases. In Phase 1 the hips may be coupled and the knees may be locked, requiring no electrical power or muscle exertion by the user (whether by FNS or by the user's muscle power) prior to descent, thus providing the user an opportunity to rest between steps.

Phase 2 may be initiated by the user operating a manual switch, by a control system signal, or by other suitable means. In Phase 2, device 10 unlocks the contralateral hip and knee to allow the user to flex the contralateral hip and knee. At the same time, device 10 may continue to lock the ipsilateral hip and knee to prevent forward tilt until the contralateral leg is flexed and has cleared the step. At this point, device 10 may unlock the user's ipsilateral hip to allow forward trunk tilt (Phase 3) with balance provided by the upper extremities.

Damping of the stance knee may be applied during Phases 3 and 4 to control flexion so the leading foot can be lowered towards the step below.

The transition between controlled lowering and leg pull through may occur with weight acceptance in Phase 5, during which damping of the leading knee may absorb energy and lessens the impact of foot-floor contact. FNS to the quadriceps of the leading leg in addition to damping may prevent collapse at the knee.

Leg pull through may begin in Phase 6 with locking of the contralateral knee for body weight support, while FNS to the knee extensors may be turned off to allow the muscles to rest. The hips may be reciprocally coupled intermittently to prevent forward trunk tilt while assisting ipsilateral hip flexion through stimulation of the contralateral hip extensors. Ipsilateral knee flexion may be supplemented by the hydraulic coupling with hip flexion. Hip and knee flexors may be under FNS control or assisted by PAS 80 just prior to toe-off to ensure foot clearance and forward progression to the step below.

Once the foot clears the step, stimulation in Phase 7 may be switched from flexors to extensors in preparation for foot placement.

With the foot in place and body in an erect position in Phase 8, both knees of the brace are locked and the hips may be coupled to prevent forward trunk tilt and the sequence may repeat with Phase 1.

The absence of knee flexion following heel strike during walking may reduce the capacity for shock absorption as body weight is transferred impulsively from the femur to the tibia causing knee hyperextension. The result may be a discontinuous velocity profile during gait that is more characteristic of serial stepping than walking.

As the user's limb advancement continues during initial loading, knee flexion may reduce vertical displacement of the user's body center of mass to conserve energy. Introduction of controlled stance phase flexion through variable damping may provide a more natural loading response by smoothing the gait velocity profile, reducing the center of mass vertical displacement, and preventing excessive knee hyperextension.

The amount of knee damping may be set based on the specific user's muscle strength, weight, and preference. By way of a non-limiting example, the amount of knee damping may be set based on observed or measured impact forces on the user's body. The damping amount may also include a safety factor to prevent the knee from buckling under the user's body weight. The damping force may be successively decreased until maximum knee flexion achieved during weight acceptance approaches the physiological threshold.

### Control System

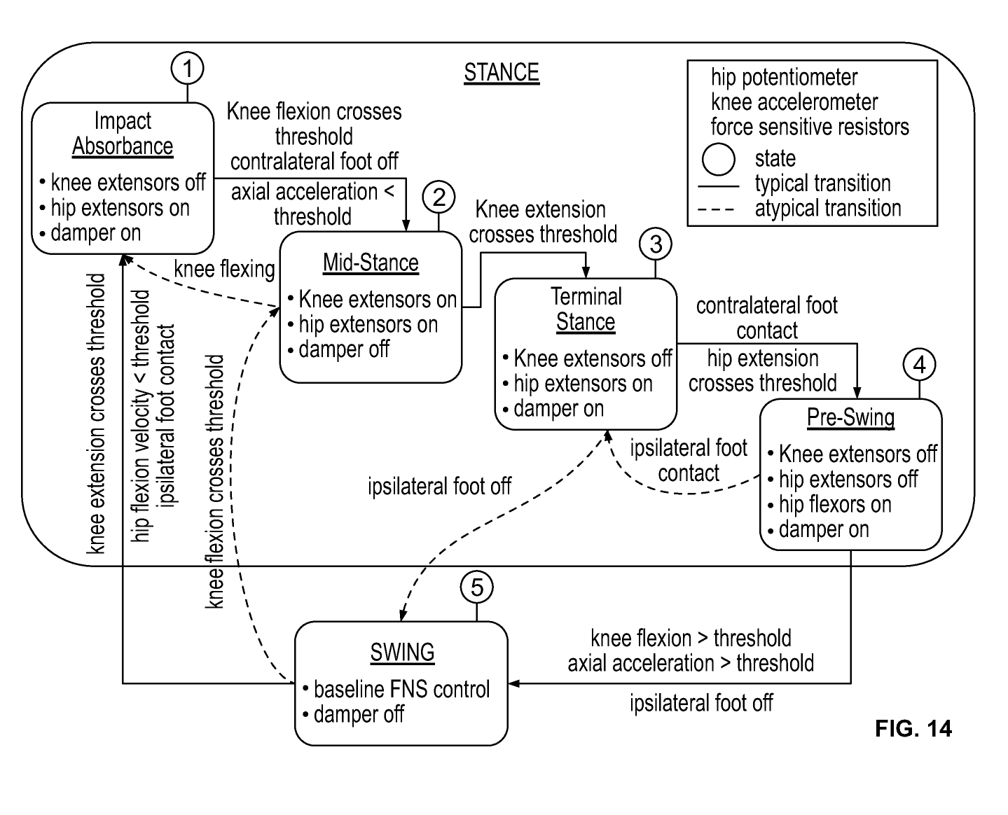

In another embodiment, as schematically represented in FIG. 14, a sensor-based controller may be added to the knee-hip device to modulate FNS to the user's knee extensor muscles to synchronize the muscle's movement with the controllable bracing provided by (and the movements allowed by) the knee-hip device. The controller described herein may be employed to control device 10 for a variety of users, including those with weakened muscles operating on their own power, those operating with help from PAS 80, those operating with help from FNS, or any combination thereof.

The controller may be configured to lock, unlock, dampen, and couple movements of the hip and knee joints to approximate normal movements, to minimize stimulation for FNS-assisted users at times when the knee-hip device is stabilizing the knee joint in the proper position, and to supplement the user's muscle power when needed to achieve a desired movement.

As shown, the controller may activate or deactivate valves in the system, and trigger FNS to particular muscles, based on conditions detected by the sensors. By way of a non-limiting example, the sensors employed in the system may include: one or more rotary or linear potentiometers may be used to measure hip angles in the sagittal plane; one or more three-axis accelerometers and gyroscopes may be employed to measure knee angle, velocity, and acceleration data; force sensitive resistors or contact sensors may be placed at the bottom of the foot or other suitable location to measure foot-ground contact at the heel and forefoot; pressure transducers to measure pressure within the hydraulic system; or force transducers to measure forces applied by the pistons.

PWM may be used to adjust current supplied to the MR damper, which adjusts the amount of damping or fluid resistance it provides. A 12 V battery, or another suitable power source, may be used to supply the current to the MR damper. A microcontroller may be used to generate and adjust the PWM control signal based on the signal given from the controller. The modulated signal may control a MOSFET switch gait that routes current to the damper. In one embodiment, the MR Damper may operate at a high frequency (about 800 Hz). The high frequency may allow for smoother control by varying the duty cycle of the PWM MOSFET gate control pulse train.

To understand the controller aspect of the invention, it may be helpful to split the typical gait cycle into a number of distinct phases. For example, impact absorbance (phase 1) occurs immediately after heel strike and is characterized by rapid knee flexion for shock absorption. As initial stance flexion ends, the knee extends during mid-stance (phase 2) to maintain for forward progression. At the conclusion of stance knee extension, the body begins to fall forward during terminal stance (phase 3). Pre-swing (phase 4) occurs near the end of stance; as the contralateral foot strikes the ground, the knee flexes in preparation for toe-off. At this point, the hip flexes causing the leg to leave the ground as the knee continues through swing flexion (phase 5). Finally, after reaching maximum flexion during swing, the knee extends until it reaches full extension and the toe touches the ground, starting the next cycle.

In one of the embodiments, a finite state controller, as illustrated in FIG. 14, may be employed to move between the different gait phases. To minimize parasitic losses of limb motion under FNS activity, the knee-hip device will remain off during both swing phases, enabling them to be lumped into a single state. Transition between the five states may proceed as follows. State 1 begins when the ipsilateral foot contacts the floor. At initial impact, the anterior alignment of the ground reaction force stabilizes the knee, allowing for knee extensor stimulation to be turned off. The damper is turned on simultaneously to absorb the shock of impact. As forward progression continues, the damper regulates knee flexion caused by thigh inertia and body weight load at the proximal end of the femur.

Once initial knee flexion passes a predetermined threshold, the controller transitions to state 2. By way of non-limiting example, the predetermined threshold may be approximately 15°. Knee and hip extensors are turned on while the damper is turned off to facilitate transfer of the body weight vector to the anterior side of the knee joint. When the knee reaches near full extension (within 5°) the FNS is turned off and the damper is turned on fully to prevent flexion and to rest the muscles as the controller move to state 3. When the contralateral foot contact is sensed, the transition to state 4 takes place. The damper is active, regulating knee flexion which occurs as the ipsilateral hip flexes and the load of weight bearing is transferred to the contralateral side. Once the ipsilateral foot leaves the ground, the damper is turned off to allow unencumbered knee motion during swing (powered by FNS, volitional effort, or assisted by PAS). Alternatively or additionally, the ipsilateral hip and knee may be coupled in early swing phase for simultaneous joint flexion to facilitate foot-floor clearance. The cycle may then repeat for continuous ambulation.

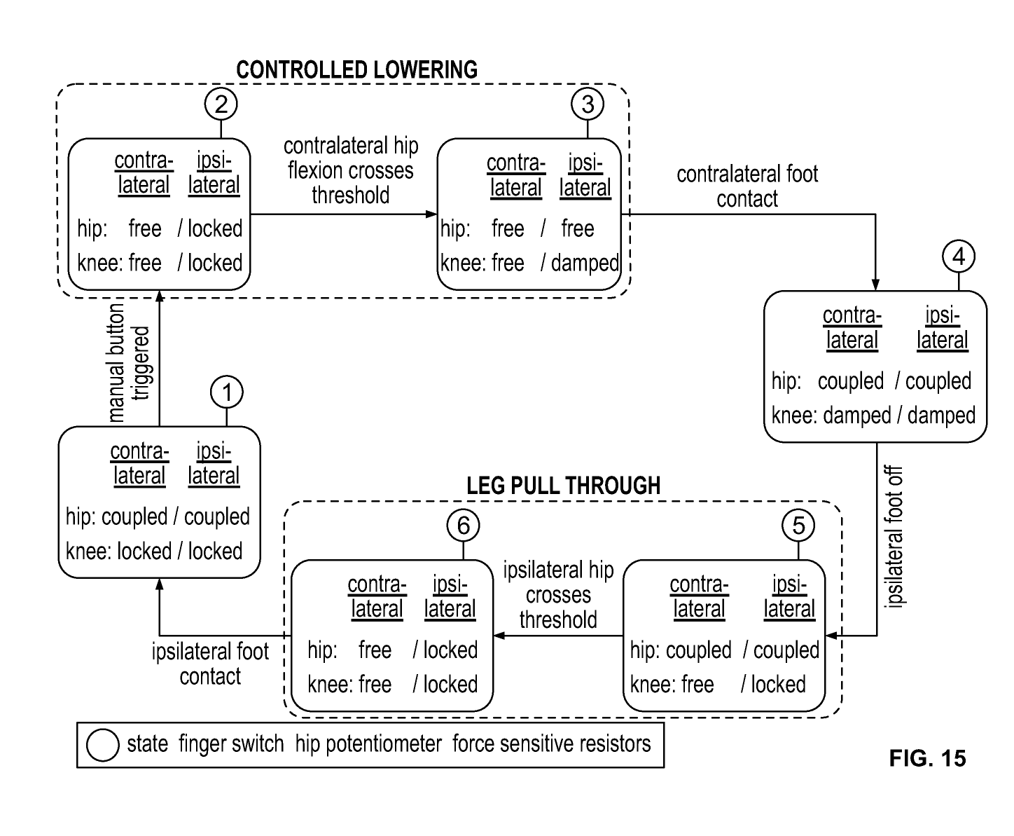

In other embodiments, the controller may be configured for different sequences of movements, such as ascending stairs or descending stairs. By way of a non-limiting example, FIG. 15 shows a schematic of a finite state controller to control device 10 during stair descent. As shown in FIG. 15, in phase 1, the hips may be coupled and the knees may be locked, requiring no electrical power or FNS prior to descent.

The user may press a manual switch or indicate intent by other means (e.g., leaning forward as detected by sensors) to begin phase 2, where the contralateral hip and knee would be unlocked to allow them to flex under FNS, volitional, or PAS 80 control. The ipsilateral hip and knee remain locked to prevent forward tilt until the contralateral leg is flexed and has cleared the step.

At this point, the ipsilateral hip is unlocked to allow the forward trunk tilt (phase 3) with balance provided by the upper extremities. Damping of the stance knee is applied during phase 3 to control flexion so the leading foot can be lowered towards the step below.

The transition between controlled lowering and leg pull through occurs with weight acceptance in phase 4, during which damping of the leading knee absorbs energy and lessens the impact of foot-floor contact.

Leg pull through begins in phase 5 with locking of the contralateral knee for body weight support while FNS to the knee extensors is turned off to allow the muscles to rest. At that phase, the hips are coupled to prevent bilateral forward trunk tilt and assist ipsilateral hip flexion with FNS of the contralateral hip extensors. Ipsilateral hip and knee flexors are under FNS, volitional, or PAS 80 control just prior to toe-off to ensure foot clearance and forward progression to the step below. Alternatively or additionally, the stance limb hip may be locked and the ipsilateral hip and knee coupled to facilitate simultaneous hip and knee flexion for pull through with the hip under FNS, voluntary or PAS control.

Once the foot clears the step, stimulation in phase 6 is switched from flexors to extensors and ankle dorsiflexors (if ankle is not mechanically fixed at neutral) in preparation for foot placement.

With the foot in place and body in an erect position stance, phase 1 is resumed on the lower step with both knees of the knee-hip device locked and the hips coupled to prevent trunk tilt. The user may rest in this position with FNS until he is ready to repeat the cycle to descend another step.

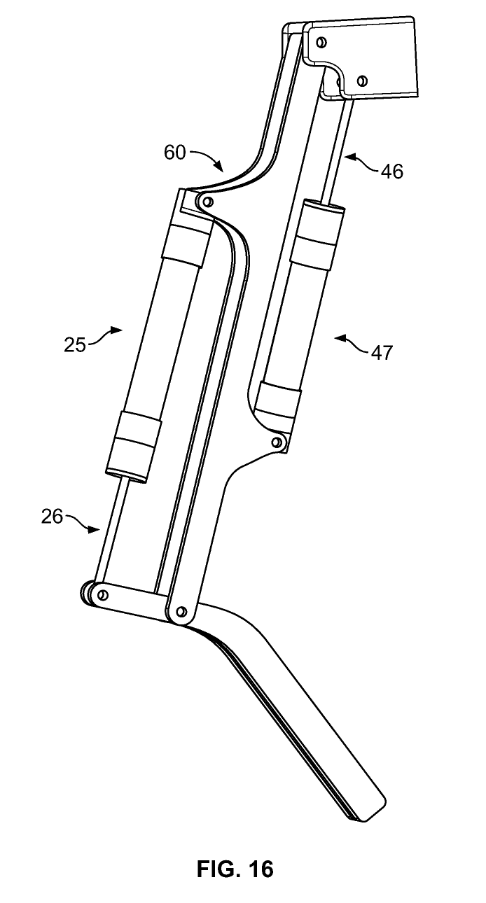

In another embodiment, as shown in FIG. 16, the blind sides of cylinder 25 of knee mechanism 20 and of cylinder 47 of the hip mechanism 40 may be mounted by clevis, or by other suitable means, on a bar 60 that runs along the user's thigh.

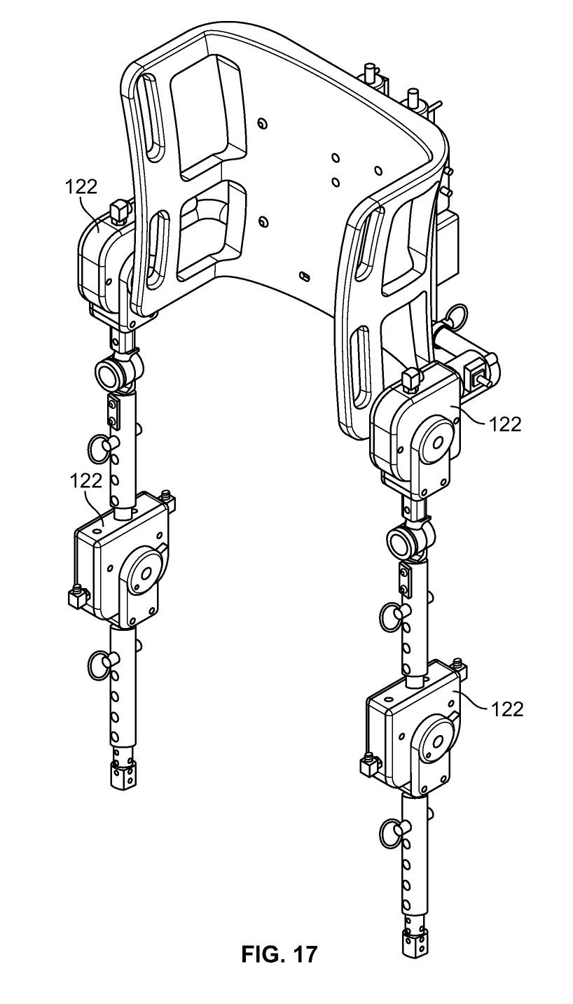

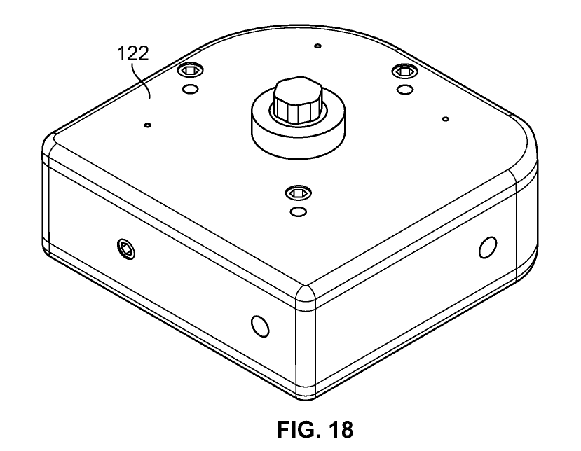

In another embodiment, compact hydraulic rotary actuators may be substituted for the pistons, (cylinders and rods) while preserving the functionality and operation described above. By way of a non-limiting example and as shown in FIGS. 17 and 18 rotary actuators 122 may be utilized with the device 10. In these embodiments, the rotary actuators 122 may be utilized with both or either of the knee mechanism 20 and hip mechanism 40. The rotary actuator 122 may be of any appropriate configuration and is not limited to that shown and described herein.

More specifically the hydraulic rotary actuators 122 may consist of a vane 125 configured to rotate around an axis. The vane 125 may sealed and may form two internal chambers 127 within a housing 129 of the rotary actuator 122. Compared to a linear actuator, this embodiment may have two primary advantages—the chambers 127 of the rotary actuator 122 may be of generally equal volume (compared to a linear cylinder, which has unequal volume rod and blind side chambers), and it may eliminate the need for a transmission. The first advantage allows for the discussed coupling to be easier to implement, as it requires an equal volume of fluid to transfer from one actuator to another. The second advantage (elimination of a transmission) potentially would result in a lighter weight embodiment compared linear cylinder implementation.

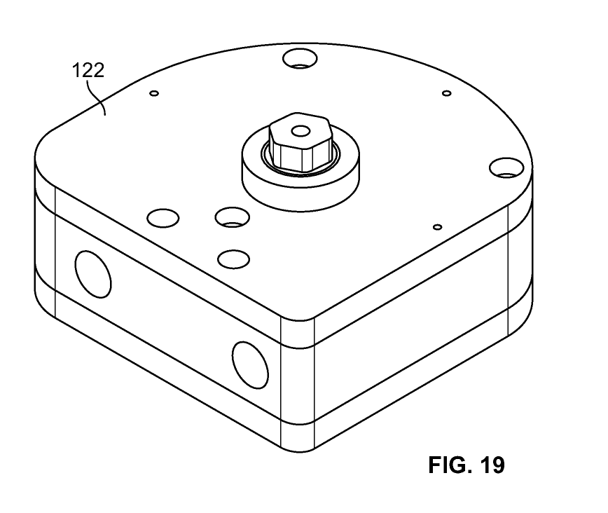

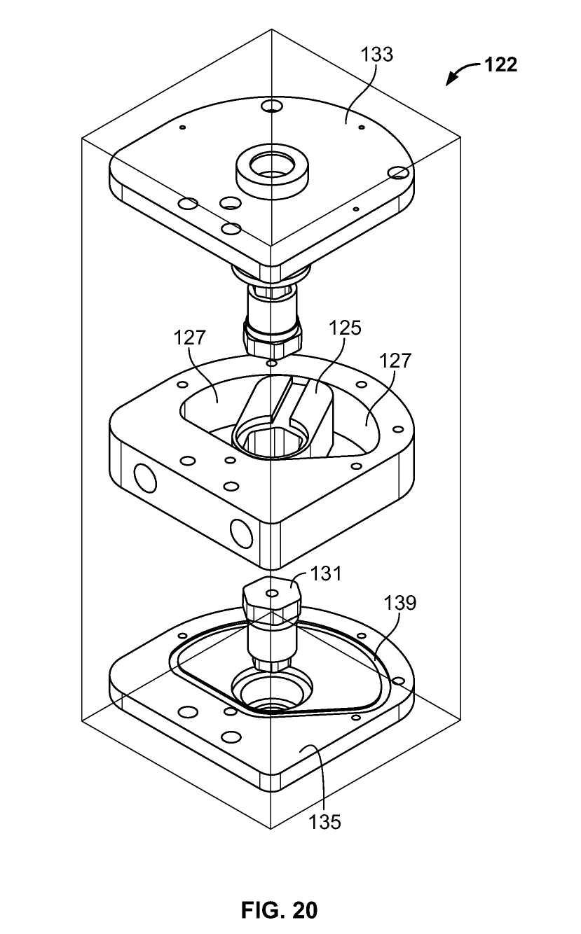

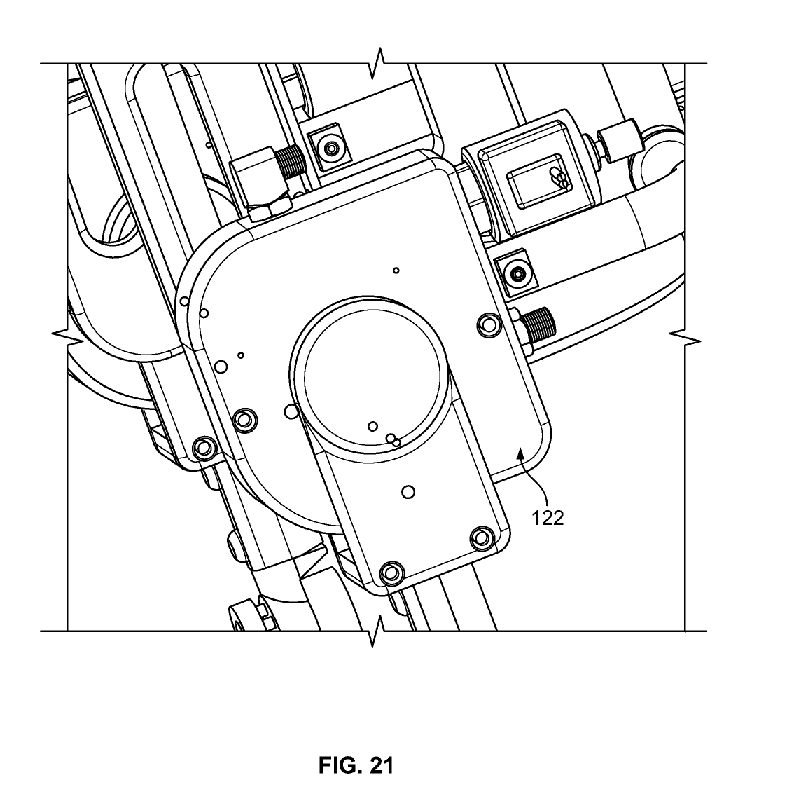

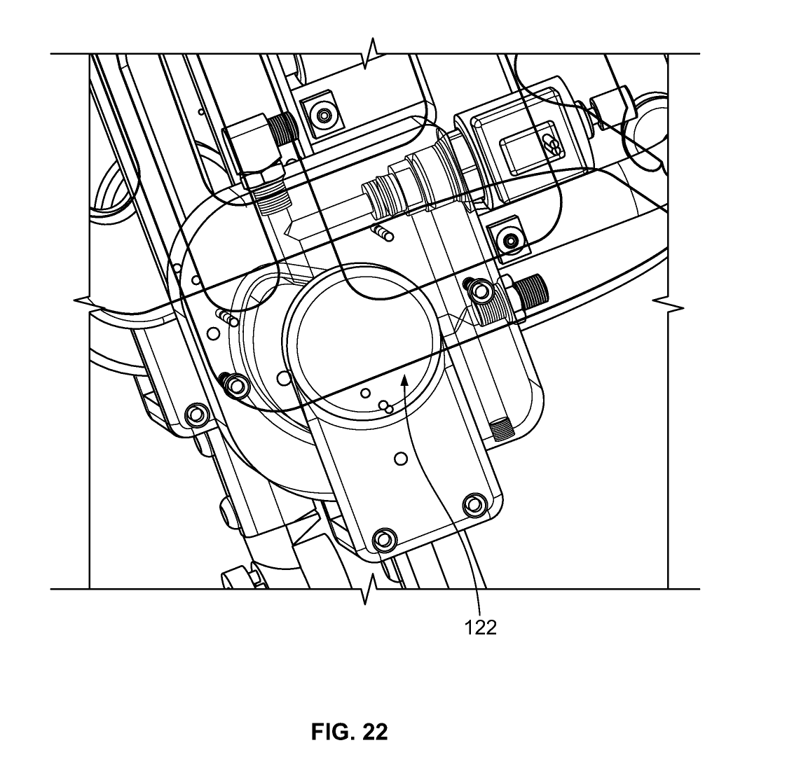

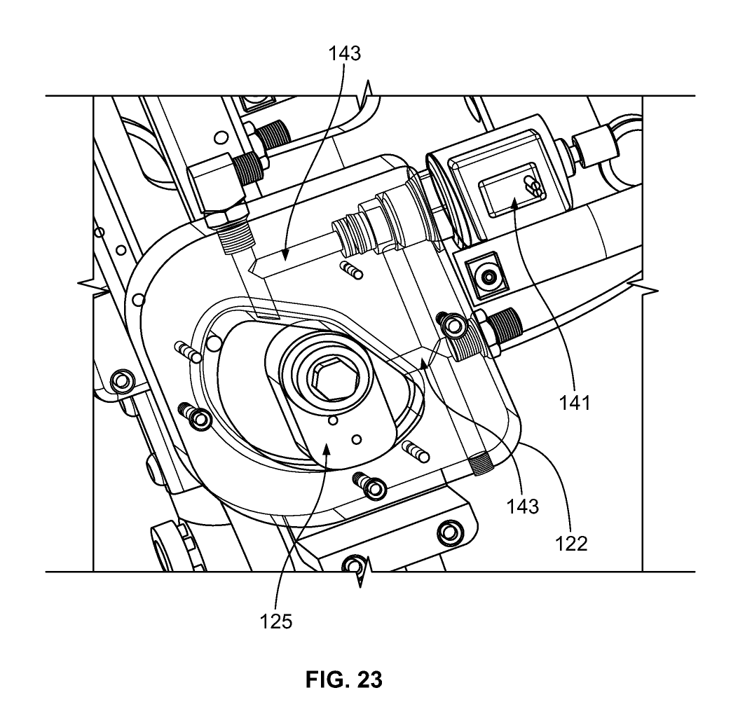

FIG. 17 shows an embodiment of this concept—instead of linear cylinders, each joint contains a rotary hydraulic actuator 122. FIGS. 18 and 19 are views of the actuator 122 by itself. Torque may be transmitted from the internal vane 125 to outside via the exposed shaft 131. FIG. 20 shows an exploded view of the rotary actuator 122, showing the internal vane 125, top and bottom faceplates 133, 135 and transmission shaft 137. Either one of or both of the faceplates 133, 135 may contain grooves 139 for rubber seals (not shown). FIG. 21 is a close up of the rotary actuator 122 mounted in the exoskeleton. FIG. 22 is a close up of the rotary actuator 122 mounted in the exoskeleton with the faceplate removed. FIG. 23 depicts the same, but with the faceplate removed and the body 129 of the rotary actuator 122 shown in phantom. In this embodiment, a two-way, two-position solenoid valve 141 (able to lock and unlock the joint) is shown integrated into the body 129, with internal flow channels 143 connecting one chamber 127 of the rotary actuator 122 to the other. The present teachings, however, are not limited to this configuration. Any appropriate configuration may be utilized without departing from the present teachings.

Although the embodiments of the present invention have been illustrated in the accompanying drawings and described in the foregoing detailed description, it is to be understood that the present invention is not to be limited to just the embodiments disclosed, but that the invention described herein is capable of numerous rearrangements, modifications and substitutions without departing from the scope of the claims hereafter. The claims as follows are intended to include all modifications and alterations insofar as they come within the scope of the claims or the equivalent thereof.

Having thus described the invention, we claim:

1. A power-assisted orthosis comprising:
a knee orthosis adapted to be secured to at least one of a user's knee joint;
a hip orthosis adapted to be secured to a user's hip joints;
a first actuator attached with the knee orthosis, the first actuator moveable in conjunction with movement of the at least one of the user's knee joint;
a second actuator attached to the hip orthosis, the second actuator moveable in conjunction with movement of the user's hip joints; and
a valve in fluid communication with a port of at least one of the first and second actuators, wherein the valve controls fluid flow within the at least one of the first and second actuators to lock or unlock at least one of the first and second actuators.

2. The power assisted orthosis of claim 1, wherein the valve is in fluid communication with both of the first and second actuators.

3. The power assisted orthosis of claim 2, wherein the valve coordinates operation of the first and second actuator permitting operation of the knee orthosis in coordination with the hip orthosis.

4. The power assisted orthosis of claim 3, wherein the coordination of operation of the knee orthosis and the hip orthosis permits at least one of standing up, sitting down, standing, walking, stair descent, stair ascent, ramp ascent, ramp descent, curb ascent, curb descent and obstacle avoidance.

5. The power assisted orthosis of claim 1, wherein the first actuator comprises a first rotary actuator or cylinder rod that extends and retracts in conjunction with movement of the at least one of the user's knee joint.

6. The power assisted orthosis of claim 5, wherein the second actuator comprises a second rotary actuator or cylinder rod that extends and retracts in conjunction with movement of the user's hip joints.

7. The power assisted orthosis of claim 1, further comprising a control system operatively coordinating the knee orthosis with the hip orthosis.

8. The power assisted orthosis of claim 7, wherein the control system includes a knee angle sensor operatively coupled with the first actuator and the valve, and a hip angle sensor operatively coupled with the second actuator and the valve.

9. The power assisted orthosis of claim 7, wherein the control system includes a pressure transducer operatively coupled with the first and second actuators to determine position or orientation of at least one of the hip or knee orthosis.

10. The power assisted orthosis of claim 7, wherein the control system includes, an inertial sensor to determine position or orientation of at least one of the hip or knee orthosis.

10. The power assisted orthosis of claim 10, wherein the inertial sensor includes at least one of an accelerometer, goniometer and magnetometer.

11. The power assisted orthosis of claim 7, wherein the control system includes a plantar pressure, foot-floor contact.

12. The power assisted orthosis of claim 7, wherein the control system includes a force sensor in at least one of the hip or knee orthosis.

13. The power assisted orthosis of claim 7, wherein the control system includes a stress/strain gauge in at least one of the hip or knee orthosis.

14. The power assisted orthosis of claim 7, wherein the valve locks/unlocks the knee orthosis dependent upon a task to be performed and based on at least one of a sensor signal input and a state of one of the hip or knee orthosis.

15. The power assisted orthosis of claim 7, wherein the valve independently locks/unlocks the hip joints of the hip orthosis dependent upon a task to be performed and based on at least one of a sensor signal input and a state of one of the hip orthosis.

16. The power assisted orthosis of claim 7, wherein the valve reciprocally couples the hip joints of the hip orthosis, or functionally couple motion of the hip joints and the at least one user's knee joint dependent upon a task to be performed and based on at least one of a sensor signal input and a state of one of the hip orthosis.

17. The power assisted orthosis of claim 7, further comprising:
an electrical stimulator;
at least one electrode operatively coupled with the electrical stimulator and in operative communication with at least one nerve of a user, wherein the electrical stimulator is adapted to provide an electrical current to stimulate functional movement of the user.

18. The power assisted orthosis of claim 17, wherein flexion of the hip joints is initiated by the electrical stimulator, and stimulation is modulated appropriately for a desired task based on at least one of a sensor signal input, stimulation history and state of the hip orthosis.

19. The power assisted orthosis of claim 17, wherein the control system further coordinates the first and second actuators with current, prior and anticipated stimulation.

20. The power assisted orthosis of claim 7 further comprising:
a proportional valve controlling fluid flow to the knee orthosis.

21. The power assisted orthosis of claim 20, wherein the control system modulates damping of the at least one of the user's knee joint for a task being performed depending on sensor input, and current/past/predicted status of the knee orthosis.

22. A power-assisted orthosis comprising:
a knee orthosis adapted to be secured to at least one of a user's knee joint;
a hip orthosis adapted to be secured to a user's hip joints;
a first actuator attached with the knee orthosis, the first actuator moveable in conjunction with movement of the at least one of the user's knee joint;
a second actuator attached to the hip orthosis, the second actuator moveable in conjunction with movement of the user's hip joints;
a motor operatively coupled with the first and second actuators;
a pump in operative communication with the motor;
a reservoir in communication with the pump for storage of pressurized fluid; and
a directional control valve in operative communication with the reservoir, wherein the directional control valve distributes fluid to coordinate motion of the first and second actuators and assist motion of the knee orthosis and hip orthosis.

23. A power-assisted orthosis comprising:
a knee constraint adapted to be secured to at least one of a user's knee joint; and
a back-drivable motor in operative communication with the knee constraint, wherein the back-drivable motor drives motion of the knee constraint and modulates damping of the user's knee joint during movement.

24. The power-assisted orthosis of claim 23, further comprising a transmission assembly in operative communication with the back-drivable motor, wherein the transmission assembly provides variable power assist to move the user's knee joint.

25. The power assisted orthosis of claim 23, further comprising a pair of hip constraints securable with a user's hip joints, the hip constraint in mechanical communication with the knee constraint, wherein at least one of the back-drivable motor and transmission assembly coordinates motion between the hip and knee constraints and/or between the pair of hip constraints.

26. A knee orthosis comprising:
an electrical stimulator;
at least one electrode operatively coupled with the electrical stimulator and in operative communication with at least one nerve of a user, wherein the electrical stimulator is adapted to provide an electrical current to stimulate functional movement of a knee of the user; and
a knee constraint adapted to operatively attach to a leg of the user, wherein the knee constraint prevents unwanted movement of the knee of the user.

27. The knee orthosis of claim 26, wherein the knee constraint is free of a motor.

28. The knee orthosis of claim 26, wherein the functional movement of the knee of the user is solely done through the electrical current.

29. The knee orthosis of claim 26, wherein the functional movement of the knee of the user is accomplished without a motor.

30. A knee orthosis comprising:
a knee constraint adapted to be secured to a user's knee joint;
an actuator attached with the knee constraint, the actuator having a rotary actuator or cylinder rod that extends and retracts in conjunction with movement of the user's knee joint;
a first valve in fluid communication with a port of the actuator, wherein the first valve controls fluid flow within the actuator to lock or unlock the cylinder rod or rotary actuator; and
a second valve or damper secured with the knee constraint, the damper or second valve configured to provide variable resistance to knee flexion and extension during movements of the user's knee joint.

31. The knee orthosis of claim 30, wherein the damper includes a magnetorheological fluid linear damper.

32. The knee orthosis of claim 30, wherein the damper includes a non-magnetorheological hydraulic fluid and a valve adapted to vary flow based on an adjustable current supplied by a pulse width modulation.

33. The knee orthosis of claim 30, wherein the damper includes a motor and transmission operatively coupled in tandem.

34. The knee orthosis of claim 33, further comprising a hydraulic piston and valve configured for a fixed, continuous level of damping.

35. The knee orthosis of claim 34, wherein the motor is adapted to provide variable damping tuned for each intended task.

36. The knee orthosis of claim 35, wherein the motor provides resistive torque to the knee constraint.

37. The knee orthosis of claim 36, further comprising a software program operating the motor to control the resistive torque applied.

38. The knee orthosis of claim 30, wherein the damper comprises a motor and transmission configured to continuously vary a coupling ratio between the user's knee joint and its ipsilateral hip.

39. A hip orthosis comprising:
a hip constraint adapted to operatively fit a user's hip;
a conversion device to convert rotatory motion of flexion and extension of the user's hip to a linear motion; and
a sensor in operative communication with the conversion device, the sensor modulated to a plurality of independent states during a gait of a user.

40. The hip orthosis of claim 39, wherein the plurality of independent states include: reciprocally coupled; freed; bi-directionally locked; and uni-directionally locked.

41. The hip orthosis of claim 39, further comprising a fluid actuator operatively coupled with the conversion device and hip constraint, wherein the fluid actuator provides motion to a user's hips.

42. The hip orthosis of claim 39, further comprising a valve in operative communication with the conversion device, the valve operatively positioning the fluid actuator to move the user's hips in a predetermined manner.

43. A hip orthosis comprising:
a hip constraint adapted to operatively fit a user's hip;
an actuator in operative communication with the hip constraint;
a pump in operative communication with the actuator; and
a bi-directional control valve adapted to pressurize the actuator to cause movement of the actuator, wherein movement of the actuator moves the hip constraint in a predetermined manner moving the user's hip.

44. A power-assisted orthosis comprising:
a knee constraint adapted to be secured to at least one of a user's knee joint;
a hip constraint adapted to be secured to a user's hip joint;
a first actuator attached with the knee constraint, the first actuator controlling movement of the knee constraint;
a second actuator attached to the hip constraint, the second actuator controlling movement of the hip constraint;
a valve in fluid communication with the first and second actuators, wherein the valve controls fluid flow within the first and second actuators to control movement the first and second actuators;
an electrical stimulator in operative communication with at least one nerve of a user, wherein the electrical stimulator is adapted to provide an electrical current to stimulate functional movement of the user's knee joint, hip joint or both; and
a controller operatively coupled with the electrical stimulator and the valve to lock, unlock, dampen, and/or couple movements of the user's hip and knee joints to approximate normal movements.

45. The power assisted orthosis of claim 44, wherein the controller minimizes stimulation from the electrical stimulator at times when the knee constraint is stabilizing the user's knee joint in its proper position.

46. The power assisted orthosis of claim 44, wherein the controller supplements a user's muscle power when needed to achieve a desired movement.
